(19) United States
(12) Patent Application Publication
(10) Pub. No.: US 2024/0043807 A1
(43) Pub. Date: Feb. 8, 2024

(54) METHODS AND COMPOSITIONS FOR DIFFERENTIATING SKELETAL MUSCLE

(71) Applicant: iXCells Biotechnologies USA, Inc., San Diego, CA (US)

(72) Inventors: Lingyan ZHANG, San Diego, CA (US); Minqing RONG, San Diego, CA (US); Nianwei LIN, San Diego, CA (US)

(21) Appl. No.: 18/258,781

(22) PCT Filed: Dec. 20, 2021
(86) PCT No.: PCT/US2021/064441

§ 371 Date: Jun. 21, 2023

Related U.S. Application Data
(60) Provisional application No. 63/129,338, filed on Dec. 22, 2020.

(51) Int. Cl. C12N 5/077

(52) U.S. Cl. CPC C12N 5/0658; C12N 2500/84; C12N 2506/45; C12N 2501/11; C12N 2501/33; C12N 2501/727; C12N 2501/115; C12N 2501/12; C12N 2501/105; C12N 2501/135

(57) ABSTRACT

Provided herein are methods and compositions for differentiating pluripotent cells, In embodiments, pluripotent cells are differentiated to form myoblast precursor cells, In embodiments, myoblast precursor cells are differentiated to form myoblasts. In embodiments, myoblasts are cultured to form myotubes. Media for culturing cells, cell products, and uses thereof are also described herein.

### CROSS-REFERENCE TO RELATED APPLICATION

This application claims the benefit of U.S. Provisional Application No. 63/129,338, filed Dec. 22, 2020, which is incorporated herein by reference in its entirety and for all purposes.

### BACKGROUND OF THE INVENTION

Differentiation of skeletal muscle is a highly controlled, multistep process, during which single muscle cells initially freely divide and then align and fuse to form multinucleated myotubes. This process of muscle differentiation in vivo is governed by a complex interplay of a wide range of growth and trophic factors, which has proven difficult and costly to reproduce in vitro.

A large number of recreational and professional athletic injuries involve skeletal muscle. Musculoskeletal disorders and diseases are also a leading cause of disability in the United States and account for more than one-half of all chronic conditions in people over 50 years of age in developed countries. Among various musculoskeletal injuries, soft tissue skeletal muscle injuries often cause a significant loss of flexibility and strength. Incomplete healing of these injuries could lead to a frequent reinjury of skeletal muscles. Therapies to improve functional recovery and shorten rehabilitation may both optimize performance and minimize morbidity. Further research is ongoing to refine these muscle-based tissue engineering applications.

### SUMMARY OF THE INVENTION

In view of the above, there is a need for effective in vitro systems for differentiating skeletal muscle, for uses such as studying models of disease conditions, screening therapeutic agents, preparing transplantable tissue, and preparing food products. Methods, systems, and compositions disclosed herein provide solutions to these needs, and other advantages as well.

In some aspects, the present disclosure provides a method of differentiating pluripotent cells. In embodiments, the method comprises culturing pluripotent cells in a culture medium including one or more of a fetal bovine serum (FBS), a chick embryo extract, and a fetuin; wherein the differentiation produces myoblast precursor cells. In embodiments, the method further comprises culturing the myoblast precursor cells in a second culture medium, wherein the second culture medium comprises one or more of a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF); wherein the culturing in the second culture medium produces myoblasts. In embodiments, the method further comprises culturing the myoblasts in a third culture medium, wherein the third culture medium comprises one or more of an insulin, ascorbic acid, oncostatin, and necrosulfonamide; wherein the culturing in the third culture medium produces myotubes. In embodiments, culturing the myotubes comprises a plurality of cycles, each cycle comprising (a) culturing the myotubes in the third culture medium, and (b) culturing the myotubes in the second culture medium.

In some aspects, the present disclosure provides myotubes produced according to methods as disclosed herein, including embodiments thereof.

In some aspects, the present disclosure provides a pharmaceutical composition including the myotubes as disclosed herein, including embodiments thereof, and a pharmaceutically acceptable carrier. In some aspects, the present disclosure provides a use of the myotubes disclosed herein in the production of a pharmaceutical composition.

In some aspects, the present disclosure provides a use of the myotubes as disclosed herein, including embodiments thereof, in the production of an edible composition.

In some aspects, the present disclosure provides a culture medium for differentiating pluripotent cells. In embodiments, the medium comprises a fetal bovine serum (FBS), a chick embryo extract, and a fetuin. In embodiments, the culture medium further comprises pluripotent cells or myoblast precursor cells differentiated therefrom.

In some aspects, the present disclosure provides a culture system for differentiating pluripotent cells, the culture system comprising: (a) a first culture medium including one or more of a fetal bovine serum (FBS), a chick embryo extract, and a fetuin; (b) a second culture medium including one or more of a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF); and (c) a third culture medium including one or more of an insulin, ascorbic acid, oncostatin, and necrosulfonamide. In embodiments, the first culture medium comprises pluripotent cells or myoblast precursor cells differentiated therefrom. In embodiments, the second culture medium comprises myoblast precursor cells or myoblasts differentiated therefrom. In embodiments, the third culture medium comprises myoblasts or myotubes differentiated therefrom.

### BRIEF DESCRIPTION OF THE DRAWINGS

FIG. 1 shows illustrative fluorescence microscopy pictures of myoblasts stained with Desmin. Satellite like progenitors were generated in cultures with either horse serum (HS) in the culture medium (top panel) or with fetal bovine serum (FBS) and chick embryo extract in the culture medium (bottom panel). Scale bars shown: 100 μm.

FIG. 2 shows illustrative fluorescence microscopy pictures of myoblasts stained with MHC. Satellite like progenitors were generated in cultures with either horse serum (HS) in the culture medium (top panel) or with fetal bovine serum (FBS) and chick embryo extract in the culture medium (bottom panel). Scale bars shown: 100 μm.

FIG. 3 shows illustrative microscopy pictures of myoblasts. Satellite like progenitors were generated in cultures with either horse serum (HS) in the culture medium (top panel) or with fetal bovine serum (FBS) and chick embryo extract in the culture medium (bottom panel). Scale bars shown: 200 μm.

FIG. 4 shows illustrative microscopy pictures of single induced pluripotent stem cells (iPSC) seeded in a medium according to an embodiment. Cells were cultured in 6-well plates that were freshly coated with collagen I prior to culturing (top panel), or in 6-well plates pre-coated with collagen I (CORNING© BIOCOAT plate). Images were taken after four days of culture, and are shown at 20× magnification.

FIG. 5 shows illustrative microscopy pictures of myotubes obtained from iPSC-derived myoblasts that were seeded in 48-well plates that were freshly coated with collagen I, and in a myotube differentiation medium according to an embodiments. Images were taken at days 6, 15, and 21 after the first addition of myotube differentiation medium, under “pulse” (changes in medium compositions according to an embodiment) or “non pulse” (no changes in medium) conditions. Scale bars shown: 100 μm.

FIG. 6 shows illustrative fluorescence microscopy pictures of fixed myotubes stained with MHC at 7 or 21 days after the first addition of a myotube differentiation medium, obtained from iPSC-derived myoblasts cultured under “pulse” or “non pulse conditions”. The “pulse” conditions improved MHC expression at the 21-day time point. Scale bars shown: 100 μm.

FIG. 7 shows illustrative microscopy pictures of myotubes obtained from iPSC-derived myoblasts, 21 days after the first addition of myotube differentiation medium according to a “pulsing” procedure, and further cultured for 7 days in a motor neuron medium, with (bottom panel) or without (top panel) co-culture with motor neurons.

FIG. 8 shows an illustrative workflow for directing differentiation of iPSCs into skeletal muscle cells (satellite-like cells, myoblasts, and myotubes), in accordance with an embodiment.

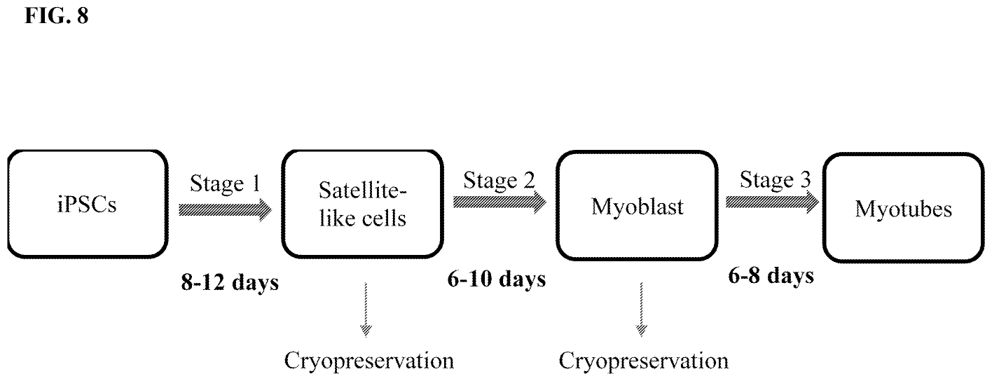

### DETAILED DESCRIPTION OF THE INVENTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Another type of pluripotent cells are induced pluripotent stem cells (iPSCs). In general, iPSCs are the product of reprogramming a non-pluripotent cell (e.g., an adult somatic cell) to a pluripotent state. A variety of methods for reprogramming non-pluripotent cells to form iPSCs are available, such as those disclosed in US2009068742 and US20170073643.

As used herein, the term “myotube” refers to a fiber structure formed by the fusion of two or more myoblasts, which possesses the structural and functional features exhibited by a naturally-occurring myotube. In some embodiments, a myotube further possesses at least one structural or functional feature that distinguishes it from naturally-occurring myotubes. Naturally-occurring mature myotubes are generally large and branched and have multiple nuclei. The fibers often have striations, and may have a functional sarcomeric organization. For example, they may have periodic distribution of sarcomeric proteins (e.g., Titin, fast MyHC) and may twitch spontaneously. In some embodiments, myotubes exhibit a fast-twitch. In some embodiments, myotubes exhibit a slow-twitch.

As used herein the term “myoblast” refers to a cell that possesses the structural and functional features exhibited by a naturally-occurring myoblast, and may or may not possess at least one structural or functional feature that distinguishes it from a naturally-occurring myoblast. Myoblasts may be identified by characteristic morphology and/or expression of one or more markers. In embodiments, the myoblasts are characterized by expression of one or both of MyoD and Desmin.

As used herein, the term “myoblast precursor” refers to a non-pluripotent cell that has the ability to differentiate into a myoblast. An example of a myoblast precursor is a satellite cell or a satellite-like cell. In general, the term “satellite cell” refers to a cell that possesses the structural and functional features exhibited by a naturally-occurring satellite cell. The term “satellite like cell” refers to a satellite cell that also possesses at least one structural or functional feature that distinguishes it from a naturally occurring satellite cells. Satellite cells and satellite-like cells may be identified by morphology and/or expression of one or more markers that are characteristic of satellite cells. In some embodiments, satellite-like cells are characterized by the expression of one or more (or all) of Pax3, Pax7, CD56/NCAM, Nanog, POU5F1/Oct-4, and SSEA-4. In embodiments, the satellite-like cells express PAX3 and PAX7. In some embodiments, the satellite-like cells express CD56/NCAM.

As used herein, the terms “inhibitor,” “repressor,” “antagonist,” and “downregulator” refer to a substance capable of detectably decreasing the expression or activity of a given gene or protein. In embodiments, the inhibitor decreases expression or activity 10%, 20%, 30%, 40%, 50%, 60%, 70%, 80%, 90% or more in comparison to a control in the absence of the inhibitor. In certain instances, expression or activity is 1.5-fold, 2-fold, 3-fold, 4-fold, 5-fold, 10-fold or lower than the expression or activity in the absence of the inhibitor.

As used herein, the terms “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable.

Genes and proteins encoded thereby are identified herein with reference to corresponding gene symbols. Additional information relating to recited gene designations, including sequence information (e.g., DNA, RNA, and amino acid sequences), aliases of genes commonly identified by shorter gene symbols, and the like are available in publicly accessible databases known to those skilled in the art, such as databases available from the National Center for Biotechnology Information (www.ncbi.nlm.nih.gov/), including GenBank (www.ncbi.nlm.nih.gov/genbank/) and the NCBI Protein database (www.ncbi.nlm.nih.gov/protein/), and UniProt (www.uniprot.org). In embodiments, the protein is a human protein.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

### Methods for Differentiating Pluripotent Cells

In some aspects, the present disclosure provides methods of differentiating pluripotent cells, such as induced pluripotent stem cells (iPSCs). In embodiments, the method comprises culturing pluripotent cells in a culture medium including one or more of a fetal bovine serum (FBS), a chick embryo extract (CEE), and a fetuin; wherein the differentiation produces myoblast precursor cells. In embodiments, the culture medium comprises at least two of the FBS, the CEE, and the fetuin. In embodiments, the medium comprises the FBS, the CEE, and the fetuin. In embodiments, one or both of the FBS and the CEE are omitted from media for one or more subsequent differentiation steps. It was surprisingly found that myoblast precursor cells obtained from culturing pluripotent cells in medium disclosed here grew faster, exhibited a significantly reduced differentiation time (e.g., about 20% to about 30%), and were easier to attach on collagen-coated vessels to reduce experimental variation, as compared to medium according to previous methods (e.g., medium instead comprising horse serum).

FBS (also known as fetal calf serum (FCS)) is the liquid fraction of clotted blood from fetal calves, typically depleted of cells, fibrin and clotting factors, but containing a large number of nutritional and macromolecular factors for supporting cell growth. Bovine serum albumin is a major component of FBS. FBS also contains growth factors, and a variety of small molecules like amino acids, sugars, lipids, and hormones. FBS is frequently used in cell cultures, and is available from a variety of commercial sources. In some embodiments, the FBS is a charcoal-stripped FBS. In general, charcoal-stripping involves contacting the FBS with activated carbon. Charcoal-stripped FBS is also commercially available, such as from R&D SYSTEMS (Catalog #S11650), THERMOFISHER (Catalog #A3382101), and SIGMA-ALDRICH (Catalog #F6765). In embodiments, the FBS is present in the medium at a concentration of about 1% to about 10%, or about 3% to about 7%. In embodiments, the FBS is present at a concentration of about or more than about 1%, 2%, 3%, 4%, 5%, 6%, 7%,8%, 9%, 10%, or 15%. In embodiments, the FBS is present at a concentration of about or at least about 1%. In embodiments, the FBS is present at a concentration of about or at least about 3%. In embodiments, the FBS is present at a concentration of about or at least about 5%.

CEE is a medium component prepared from whole chicken embryos. Procedures for the preparation of CEE are known in the art (see, e.g., Pajtler et al., J Vis Exp. 2010; (45): 2380). CEE is also commercially available from a variety of sources, such as from MP BIOMEDICALS (SKU #092850145), GEMINI BIO-PRODUCTS (Catalog #100-163P), and USBIOLOGICAL (Catalog #C3999). In embodiments, the CEE is present in the culture medium at a concentration of about 0.1% to about 5%, 0.1% to about 1%, or 0.3% to about 0.7%. In embodiments, the CEE is present at a concentration of about or more than about 0.1%, 0.2%, 0.3%, 0.4%, 0.5%, 0.6%, 0.7%, 0.8%, 0.9%, 1%, 1.5%, or 2%. In embodiments, the CEE is present at a concentration of about or at least about 0.1%. In embodiments, the CEE is present at a concentration of about or at least about 0.3%. In embodiments, the CEE is present at a concentration of about or at least about 0.5%.

Fetuin is an abundant glycosylated serum protein, which functions as a transport and storage protein. It belongs to statin family and is primarily synthesized in the liver. In embodiments, the fetuin is a bovine fetuin. Bovine fetuine can be isolated from naturally occurring sources (e.g., FBS), or may be recombinantly produced. Fetuin is also available from a variety of commercial sources, such as VWR INTERNATIONAL (Catalog No. IC10487401), SIGMA-ALDRICH (Catalog No. F3004), and BIO-RAD (Product Code 4430-2204). In embodiments, the FBS is a charcoal-stripped FBS, and the fetuin is a bovine fetuin. In embodiments, the fetuin is present in the culture medium at a concentration of about 1 mg/mL to about 100 mg/mL, about 20 mg/mL to about 80 mg/mL, or about 30 mg/mL to about 70 mg/mL. In embodiments, the fetuin is present at a concentration of about or more than about 10 mg/mL, 20 mg/mL, 30 mg/mL, 40 mg/mL, 50 mg/mL, 60 mg/mL, 70 mg/mL, 80 mg/mL, 90 mg/mL or 100 mg/mL. In embodiments, the fetuin is present at a concentration of about or at least about 10 mg/mL. In embodiments, the fetuin is present at a concentration of about or at least about 30 mg/mL. In embodiments, the fetuin is present at a concentration of about or at least about 50 mg/mL.

In embodiments, (a) the FBS is present at a concentration of about 1% to about 10%; (b) the chick embryo extract is present at a concentration of about 0.1% to about 5%; and/or (c) the fetuin is present at a concentration of about 10 ng/mL to about 100 ng/mL.

In embodiments, the culture medium further includes one or more of (e.g., 2, 3, 4, 5, or all of) an epidermal growth factor (EGF), an insulin, dexamethasone, a ROCK inhibitor, ascorbic acid, a basic fibroblast growth factor (bFGF), a GSK3 inhibitor, or an ALK5 inhibitor.

In embodiments, the culture medium comprises an EGF, such as human EGF. In embodiments, the EGF is from a recombinant source. In embodiments, the EGF is a recombinantly produced human EGF (hr-EGF). In embodiments, the EGF is present in the culture medium at a concentration of about 1 ng/mL to about 100 ng/mL, about 1 ng/mL to about 50 ng/mL, or about 5 ng/mL to about 15 ng/mL. In embodiments, the EGF is present at a concentration of about or more than about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 15 ng/mL, or 20 ng/mL. In embodiments, the EGF is present at a concentration of about or at least about 1 ng/mL. In embodiments, the EGF is present at a concentration of about or at least about 5 ng/mL. In embodiments, the EGF is present at a concentration of about or at least about 10 ng/mL.

In embodiments, the culture medium comprises an insulin, such as a human insulin. In embodiments, the insulin is from a recombinant source. In embodiments, the insulin is a recombinantly produced human insulin. In embodiments, the insulin is present in the culture medium at a concentration of about 1 μg/mL to about 100 μg/mL, about 1 μg/mL to about 50 μg/mL, or about 5 μg/mL to about 15 μg/mL. In embodiments, the insulin is present at a concentration of about or more than about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 15 μg/mL, or 20 μg/mL. In embodiments, the insulin is present at a concentration of about or at least about 1 μg/mL. In embodiments, the insulin is present at a concentration of about or at least about 5 μg/mL. In embodiments, the insulin is present at a concentration of about or at least about 10 μg/mL.

In embodiments, the culture medium comprises dexamethasone. In embodiments, the dexamethasone is present in the culture medium at a concentration of about 10 ng/mL to about 1000 ng/mL, about 100 ng/mL to about 750 ng/mL, or about 300 ng/mL to about 500 ng/mL. In embodiments, the dexamethasone is present at a concentration of about or more than about 50 ng/mL, 100 ng/mL, 200 ng/mL, 300 ng/mL, 400 ng/mL, 500 ng/mL, 600 ng/mL, 700 ng/mL, or 800 ng/mL. In embodiments, the dexamethasone is present at a concentration of about or at least about 100 ng/mL. In embodiments, the dexamethasone is present at a concentration of about or at least about 200 ng/mL. In embodiments, the dexamethasone is present at a concentration of about or at least about 400 ng/mL.

In embodiments, the culture medium comprises a ROCK inhibitor. Rho associated kinases (ROCK) are serine/threonine kinases that serve as downstream effectors of Rho (e.g., the isoforms RhoA, RhoB and RhoC). Non-limiting examples of ROCK inhibitors include polynucleotides, polypeptides, and small molecules. A ROCK inhibitor may decrease ROCK expression and/or ROCK activity. Illustrative examples of ROCK inhibitors contemplated herein include, but are not limited to, dominant negative ROCK variants, siRNA, shRNA, miRNA and antisense nucleic acids that target ROCK. In embodiments, the ROCK inhibitor is a small molecule. Several ROCK inhibitors are known in the art. Non-limiting examples of ROCK inhibitors include thiazovivin, Y27632, Fasudil, AR122-86, Y27632 H-1152, Y-30141, Wf-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, and (R)-(+)-trans-N-(4-Pyridyl)-4-(1-aminoethyl)-cyclohexanecarboxamide, and ROCK inhibitors disclosed in U.S. Pat. No. 8,044,201, which is herein incorporated by reference in its entirety. In embodiments, the ROCK inhibitor is Y27632. In embodiments, the ROCK inhibitor is present in the culture medium at a concentration of about 1 μM to about 100 μM, about 1 μM to about 50 μM, or about 5 μM to about 15 μM. In embodiments, the ROCK inhibitor is present at a concentration of about or more than about 1 μM, 2 μM, 3 μM, 4 μM, 5 μM, 6 μM, 7 μM, 8 μM, 9 μM, 10 μM, 15 μM, or 20 μM. In embodiments, the ROCK inhibitor is present at a concentration of about or at least about 1 μM. In embodiments, the ROCK inhibitor is present at a concentration of about or at least about 5 μM. In embodiments, the ROCK inhibitor is present at a concentration of about or at least about 10 μM.

In embodiments, the culture medium comprises ascorbic acid (also known as vitamin C). In embodiments, the ascorbic acid is present in the culture medium at a concentration of about 1 μg/mL to about 100 μg/mL, about 20 μg/mL to about 80 μg/mL, or about 30 μg/mL to about 70 μg/mL. In embodiments, the ascorbic acid is present at a concentration of about or more than about 10 μg/mL, 20 μg/mL, 30 μg/mL, 40 μg/mL, 50 μg/mL, 60 μg/mL, 70 μg/mL, 80 μg/mL, 90 μg/mL or 100 μg/mL. In embodiments, the ascorbic acid is present at a concentration of about or at least about 10 μg/mL. In embodiments, the ascorbic acid is present at a concentration of about or at least about 30 μg/mL. In embodiments, the ascorbic acid is present at a concentration of about or at least about 50 μg/mL.

In embodiments, the culture medium comprises a basic fibroblast growth factor (such as a human bFGF). In embodiments, the bFGF is from a recombinant source. In embodiments, the bFGF is a recombinantly produced human bFGF. In embodiments, the bFGF is present in the culture medium at a concentration of about 0.1 ng/mL to about 10 ng/mL, about 0.1 ng/mL to about 5 ng/mL, or about 0.5 ng/mL to about 1.5 ng/mL. In embodiments, the bFGF is present at a concentration of about or more than about 0.1 ng/mL, 0.2 ng/mL, 0.3 ng/mL, 0.4 ng/mL, 0.5 ng/mL, 0.6 ng/mL, 0.7 ng/mL, 0.8 ng/mL, 0.9 ng/mL, 1 ng/mL, 1.5 ng/mL, or 2 ng/mL. In embodiments, the bFGF is present at a concentration of about or at least about 0.1 ng/mL. In embodiments, the bFGF is present at a concentration of about or at least about 0.5 ng/mL. In embodiments, the bFGF is present at a concentration of about or at least about 1 ng/mL.

In embodiments, the culture medium comprises a GSK3 inhibitor. GSK3 is a protein-serine/threonine kinase that phosphorylates a broad range of substrates including glycogen synthase, several transcription factors, and translation initiation factors. GSK3 is involved in multiple cellular processes including metabolism, cell survival, proliferation, and differentiation. Non-limiting examples of GSK3 inhibitors include polynucleotides, polypeptides, and small molecules. A GSK3 inhibitor may decrease GSK3 expression and/or GSK3 activity. Illustrative examples of GSK3 inhibitors contemplated herein include, but are not limited to, dominant negative GSK3 variants, siRNA, shRNA, miRNA and antisense nucleic acids that target GSK3. In embodiments, the GSK3 inhibitor is a small molecule. Several GSK3 inhibitors are known in the art. Non-limiting examples of GSK3 inhibitors include Kenpaullone, 1-Azakenpaullone, CHIR99021, CHIR98014, AR-A014418, CT99021, CT20026, SB216763, AR-A014418, lithium, TDZD-8, BIO, BIO-Acetoxime, (5-Methyl-1H-pyrazol-3-yl)-(2-phenylquinazolin-4-yl)amine, Pyridocarbazole-cyclopenadienylruthenium complex, 4-Benzyl-2-methyl-1,2,4-thiadiazolidine-3,5-dione, 2-Thio(3-iodobenzyl)-5-(1-pyridyl)-[1,3,4]-oxadiazole, OTDZT, alpha-4-Dibromoacetophenone, AR-A0144-18, 3-(1-(3-Hydroxypropyl)-1H-pyrrolo[2,3-b]pyridin-3-yl]-4-pyrazin-2-yl-pyrrole-2,5-dione, TWS119 pyrrolopyrimidine compound, L803 H-KEAPPAPPQSpP-NH2 or its myristoylated form, 2-Chloro-1-(4,5-dibromo-thiophen-2-yl)-ethanone, GF109203X, R0318220, and TIBPO. In embodiments, the GSK3 inhibitor is CHIR99021. In embodiments, the GSK3 inhibitor is present in the culture medium at a concentration of about 1 μM to about 10 μM, or about 2 μM to about 4 μM. In embodiments, the GSK3 inhibitor is present at a concentration of about or more than about 0.5 μM, 1 μM, 2 μM, 3 μM, 4 μM, 5 μM, 6 μM, 7 μM, 8 μM, 9 μM, or 10 μM. In embodiments, the GSK3 inhibitor is present at a concentration of about or at least about 0.5 μM. In embodiments, the GSK3 inhibitor is present at a concentration of about or at least about 1 μM. In embodiments, the GSK3 inhibitor is present at a concentration of about or at least about 3 μM.

In embodiments, the culture medium comprises an ALK5 inhbitor. ALK5 (also known as TGFβ receptor) is a serine/threonine protein kinase that forms a complex for transducing the TGFβ signal from the cell surface to the cytoplasm. Non-limiting examples of ALK5 inhibitors include polynucleotides, polypeptides, and small molecules. An ALK5 inhibitor may decrease ALK5 expression and/or ALK5 activity. Illustrative examples of ALK5 inhibitors contemplated herein include, but are not limited to, dominant negative ALK5 variants, siRNA, shRNA, miRNA and antisense nucleic acids that target ALK5. In embodiments, the ALK5 inhibitor is a small molecule. Several ALK5 inhibitors are known in the art. Non-limiting examples of ALK5 inhibitors include RepSox, SB431542, A-83-01 (also known as 3-(6-Methyl-2-pyridinyl)-N-phenyl-4-(4-quinolinyl)-1H-pyrazole-1-carbothioamide), 2-(3-(6-Methylpyridin-2-yl)-1H-pyrazol-4-yl)-1,5-naphthyridine, GW788388 (4-{4-[3-(pyridin-2-yl)-1H-pyrazol-4-yl]pyridin-2-yl}-N-(tetrahydro-2H-pyran-4-yl)benzamide), IN-1130 (3-((5-(6-methylpyridin-2-yl)-4-(quinoxalin-6-yl)-1H-imidazol-2-yl)methyl)benzamide), SM16, GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride), and pyrimidine derivatives such as those disclosed in US20090209539A1 (incorporated herein by reference). In embodiments, the ALK5 inhibitor is RepSox. In embodiments, the ALK5 inhibitor is present in the culture medium at a concentration of about 0.5 μM to about 10 μM, or about 1 μM to about 3 μM. In embodiments, the ALK5 inhibitor is present at a concentration of about or more than about 0.5 μM, 1 μM, 2 μM, 3 μM, 4 μM, 5 μM, 6 μM, 7 μM, 8 μM, 9 μM, or 10 μM. In embodiments, the ALK5 inhibitor is present at a concentration of about or at least about 0.5 μM. In embodiments, the ALK5 inhibitor is present at a concentration of about or at least about 1 μM. In embodiments, the ALK5 inhibitor is present at a concentration of about or at least about 2 μM.

In embodiments, the culture medium comprises an EGF, an insulin, a ROCK inhibitor, a bFGF, a GSK3 inhibitor, and an ALK5 inhibitor.

In embodiments, (a) the EGF is a human EGF, (b) the insulin is a human insulin, (c) the ROCK inhibitor is Y-27632, (d) the bFGF is a human bFGF, (e) the GSK3 inhibitor is CHIR99021, and/or (f) the ALK5 inhibitor is RepSox.

In embodiments, the pluripotent cells are induced pluripotent stem cells (iPSCs). In embodiments, the pluripotent cells are mouse or a human cells. In embodiments, the pluripotent cells are human cells. In embodiments, the cells are human iPSCs. In embodiments, the pluripotent cells are cultured in the culture medium for about 3 to about 20 days, about 5 to about 15 days, or about 8 to about 12 days. In embodiments, the pluripotent cells are cultured in the culture medium for about or more than about 5 days, 6 days, 7 days, 8 days, 9 days, 10 days, 11 days, 12 days, 13 days, 14 days, or 15 days. In embodiments, the pluripotent cells are cultured for about or at least about 8 days. In embodiments, the pluripotent cells are cultured for about or at least about 10 days. In embodiments, the pluripotent cells are cultured for about or at least about 12 days. In embodiments, the pluripotent cells are cultured for about 8 to about 12 days. In embodiments, the pluripotent cells are cultured in the culture medium for a duration sufficient to form myoblast precursor cells. In embodiments, the myoblast precursor cells comprise satellite-like cells. In embodiments, the myoblast precursor cells are characterized by expression of one or more (e.g., 2, 3, 4, or all) of Pax3, Pax7, CD56/NCAM, Nanog, POU5F1/Oct-4, and SSEA-4. In embodiments, the myoblast precursor cells expression all of Pax3, Pax7, CD56/NCAM, Nanog, POU5F1/Oct-4, and SSEA-4. In embodiments, the myoblast precursors are cryopreserved for later use, such as for differentiation into myoblasts.

In embodiments, the culture medium does not include a horse serum.

In embodiments, the pluripotent cells are cultured on a surface pretreated with collagen. In embodiments the collagen is collagen I. In embodiments, the collagen I is a rat collagen I. Culture dishes and plates pre-treated with collagen I are commercially available, such as BIOCOAT plates from CORNING. In embodiments, the culture surface is freshly treated to coat the surface with collagen I prior to use, such as within 72 hours, within 48 hours, within 24 hours, or within 12 hours of use. In embodiments, the surface is freshly treated by coating with collagen I within 24 hours of culturing the cells.

### Methods for Differentiating Myoblast Precursors

In embodiments, the methods further comprise differentiating myoblast precursor cells derived from differentiating pluripotent cells according to a method described herein. In embodiments, differentiating the myoblast precursor cells comprises culturing the myoblast precursor cells in a second culture medium to produce myoblasts. In embodiments, the second culture medium comprises one or more (e.g., 2, 3, 4, 5, or all) of a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF). In embodiments, the second culture medium comprises a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF). In embodiments, the second culture medium does not comprise one or both of FBS or CEE.

In embodiments, the second culture medium comprises a horse serum. In embodiments, the horse serum is used instead of FBS. In embodiments, the horse serum is present in the medium at a concentration of about 1% to about 10%, or about 3% to about 7%. In embodiments, the horse serum is present at a concentration of about or more than about 1%, 2%, 3%, 4%, 5%, 6%, 7%, 8%, 9%, 10%, or 15%. In embodiments, the horse serum is present at a concentration of about or at least about 1%. In embodiments, the horse serum is present at a concentration of about or at least about 3%. In embodiments, the horse serum is present at a concentration of about or at least about 5%.

In embodiments, the second culture medium comprises a fetuin, such as bovine fetuine. In embodiments, the fetuin is present in the second culture medium at a concentration of about 1 mg/mL to about 100 mg/mL, about 20 mg/mL to about 80 mg/mL, or about 30 mg/mL to about 70 mg/mL. In embodiments, the fetuin is present at a concentration of about or more than about 10 mg/mL, 20 mg/mL, 30 mg/mL, 40 mg/mL, 50 mg/mL, 60 mg/mL, 70 mg/mL, 80 mg/mL, 90 mg/mL or 100 mg/mL. In embodiments, the fetuin is present at a concentration of about or at least about 10 mg/mL. In embodiments, the fetuin is present at a concentration of about or at least about 30 mg/mL. In embodiments, the fetuin is present at a concentration of about or at least about 50 mg/mL.

In embodiments, the second culture medium comprises an EGF, such as a human EGF. In embodiments, the EGF is from a recombinant source. In embodiments, the EGF is a recombinantly produced human EGF (hr-EGF). In embodiments, the EGF is present in the second culture medium at a concentration of about 1 ng/mL to about 100 ng/mL, about 1 ng/mL to about 50 ng/mL, or about 5 ng/mL to about 15 ng/mL. In embodiments, the EGF is present at a concentration of about or more than about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 15 ng/mL, or 20 ng/mL. In embodiments, the EGF is present at a concentration of about or at least about 1 ng/mL. In embodiments, the EGF is present at a concentration of about or at least about 5 ng/mL. In embodiments, the EGF is present at a concentration of about or at least about 10 ng/mL.

In embodiments, the second culture medium comprises an insulin, such as human insulin. In embodiments, the insulin is from a recombinant source. In embodiments, the insulin is a recombinantly produced human insulin. In embodiments, the insulin is present in the second culture medium at a concentration of about 1 μg/mL to about 100 μg/mL, about 1 μg/mL to about 50 μg/mL, or about 5 μg/mL to about 15 μg/mL. In embodiments, the insulin is present at a concentration of about or more than about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 15 μg/mL, or 20 μg/mL. In embodiments, the insulin is present at a concentration of about or at least about 1 μg/mL. In embodiments, the insulin is present at a concentration of about or at least about 5 μg/mL. In embodiments, the insulin is present at a concentration of about or at least about 10 μg/mL.

In embodiments, the second culture medium comprises dexamethasone. In embodiments, the dexamethasone is present in the second culture medium at a concentration of about 10 ng/mL to about 1000 ng/mL, about 100 ng/mL to about 750 ng/mL, or about 300 ng/mL to about 500 ng/mL. In embodiments, the dexamethasone is present at a concentration of about or more than about 50 ng/mL, 100 ng/mL, 200 ng/mL, 300 ng/mL, 400 ng/mL, 500 ng/mL, 600 ng/mL, 700 ng/mL, or 800 ng/mL. In embodiments, the dexamethasone is present at a concentration of about or at least about 100 ng/mL. In embodiments, the dexamethasone is present at a concentration of about or at least about 200 ng/mL. In embodiments, the dexamethasone is present at a concentration of about or at least about 400 ng/mL.

In embodiments, the second culture medium comprises a ROCK inhibitor. Non-limiting examples of ROCK inhibitors are described herein. In embodiments, the ROCK inhibitor is Y-27632. In embodiments, the ROCK inhibitor is present in the second culture medium at a concentration of about 1 μM to about 100 μM, about 1 μM to about 50 μM, or about 5 μM to about 15 μM. In embodiments, the ROCK inhibitor is present at a concentration of about or more than about 1 μM, 2 μM, 3 μM, 4 μM, 5 μM, 6 μM, 7 μM, 8 μM, 9 μM, 10 μM, 15 μM, or 20 μM. In embodiments, the ROCK inhibitor is present at a concentration of about or at least about 1 μM. In embodiments, the ROCK inhibitor is present at a concentration of about or at least about 5 μM. In embodiments, the ROCK inhibitor is present at a concentration of about or at least about 10 μM.

In embodiments, the second culture medium comprises, ascorbic acid. In embodiments, the ascorbic acid is present in the second culture medium at a concentration of about 1 μg/mL to about 100 μg/mL, about 20 μg/mL to about 80 μg/mL, or about 30 μg/mL to about 70 μg/mL. In embodiments, the ascorbic acid is present at a concentration of about or more than about 10 μg/mL, 20 μg/mL, 30 μg/mL, 40 μg/mL, 50 μg/mL, 60 μg/mL, 70 μg/mL, 80 μg/mL, 90 μg/mL or 100 μg/mL. In embodiments, the ascorbic acid is present at a concentration of about or at least about 10 μg/mL. In embodiments, the ascorbic acid is present at a concentration of about or at least about 30 μg/mL. In embodiments, the ascorbic acid is present at a concentration of about or at least about 50 μg/mL.

In embodiments, the second culture medium comprises an ALK5 inhibitor. Non-limiting examples of ALK5 inhibitors are provided herein. In embodiments, the ALK5 inhibitor is SB431542. In embodiments, the ALK5 inhibitor is present in the second culture medium at a concentration of about 0.5 μM to about 10 μM, or about 1 μM to about 3 μM. In embodiments, the ALK5 inhibitor is present at a concentration of about or more than about 0.5 μM, 1 μM, 2 μM, 3 μM, 4 μM, 5 μM, 6 μM, 7 μM, 8 μM, 9 μM, or 10 μM. In embodiments, the ALK5 inhibitor is present at a concentration of about or at least about 0.5 μM. In embodiments, the ALK5 inhibitor is present at a concentration of about or at least about 1 μM. In embodiments, the ALK5 inhibitor is present at a concentration of about or at least about 2 μM.

In embodiments, the second culture medium comprises an HGF, such as a human HGF. In embodiments, the HGF is from a recombinant source. In embodiments, the HGF is a recombinantly produced human HGF. In embodiments, the HGF is present in the second culture medium at a concentration of about 1 ng/mL to about 100 ng/mL, about 1 ng/mL to about 50 ng/mL, or about 10 ng/mL to about 30 ng/mL. In embodiments, the HGF is present at a concentration of about or more than about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, or 50 ng/mL. In embodiments, the HGF is present at a concentration of about or at least about 5 ng/mL. In embodiments, the HGF is present at a concentration of about or at least about 10 ng/mL. In embodiments, the HGF is present at a concentration of about or at least about 20 ng/mL.

In embodiments, the second culture medium comprises a basic fibroblast growth factor (such as a human bFGF). In embodiments, the bFGF is from a recombinant source. In embodiments, the bFGF is a recombinantly produced human bFGF. In embodiments, the bFGF is present in the second culture medium at a concentration of about 1 ng/mL to about 100 ng/mL, about 1 ng/mL to about 50 ng/mL, or about 10 ng/mL to about 30 ng/mL. In embodiments, the bFGF is present at a concentration of about or more than about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, or 50 ng/mL. In embodiments, the bFGF is present at a concentration of about or at least about 5 ng/mL. In embodiments, the bFGF is present at a concentration of about or at least about 10 ng/mL. In embodiments, the bFGF is present at a concentration of about or at least about 20 ng/mL.

In embodiments, the second culture medium comprises an insulin-like growth factor 1 (such as a human IGF1). In embodiments, the IGF1 is from a recombinant source. In embodiments, the IGF1 is a recombinantly produced human IGF1. In embodiments, the IGF1 is present in the second culture medium at a concentration of about 1 ng/mL to about 100 ng/mL, about 1 ng/mL to about 50 ng/mL, or about 5 ng/mL to about 15 ng/mL. In embodiments, the EGF is present at a concentration of about or more than about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 15 ng/mL, or 20 ng/mL. In embodiments, the EGF is present at a concentration of about or at least about 1 ng/mL. In embodiments, the EGF is present at a concentration of about or at least about 5 ng/mL. In embodiments, the EGF is present at a concentration of about or at least about 10 ng/mL.

In embodiments, the second culture medium comprises an oncostatin (such as a human oncostatin). In embodiments, the oncostatin is from a recombinant source. In embodiments, the oncostatin is a recombinantly produced human oncostatin. In embodiments, the oncostatin is present in the second culture medium at a concentration of about 1 ng/mL to about 100 ng/mL, about 1 ng/mL to about 50 ng/mL, or about 10 ng/mL to about 30 ng/mL. In embodiments, the oncostatin is present at a concentration of about or more than about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, or 50 ng/mL. In embodiments, the oncostatin is present at a concentration of about or at least about 5 ng/mL. In embodiments, the oncostatin is present at a concentration of about or at least about 10 ng/mL. In embodiments, the oncostatin is present at a concentration of about or at least about 20 ng/mL.

In embodiments, the second culture medium comprises a platelet derived growth factor (such as a human PDGF). In embodiments, the PDGF is from a recombinant source. In embodiments, the PDGF is a recombinantly produced human PDGF. In embodiments, the PDGF is present in the second culture medium at a concentration of about 1 ng/mL to about 100 ng/mL, about 1 ng/mL to about 50 ng/mL, or about 10 ng/mL to about 30 ng/mL. In embodiments, the PDGF is present at a concentration of about or more than about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, or 50 ng/mL. In embodiments, the PDGF is present at a concentration of about or at least about 5 ng/mL. In embodiments, the PDGF is present at a concentration of about or at least about 10 ng/mL. In embodiments, the PDGF is present at a concentration of about or at least about 20 ng/mL.

In embodiments, the myoblast precursor cells are cultured in the second culture medium for about 3 to about 14 days, or about 6 to about 10 days. In embodiments, the myoblast precursor cells are cultured in the second culture medium for about or more than about 4 days, 5 days, 6 days, 7 days, 8 days, 9 days, 10 days, 11 days, 12 days, 13 days, or 14 days. In embodiments, the myoblast precursor cells are cultured in the second culture medium for about or at least about 6 days. In embodiments, the myoblast precursor cells are cultured in the second culture medium for about or at least about 8 days. In embodiments, the myoblast precursor cells are cultured in the second culture medium for about or at least about 10 days. In embodiments, the myoblast precursor cells are cultured in the second culture medium for a duration sufficient to form myoblasts. In embodiments, the myoblasts are characterized by expression of one or both of MyoD and Desmin. In embodiments, the myoblasts are cryopreserved for later use, such as for culturing to form myotubes.

### Methods for Culturing Myotubes

In embodiments, the methods further comprise culturing myoblasts derived from pluripotent cells according to a method described herein to form myotubes. In embodiments, culturing myoblasts to form myotubes comprises culturing the myoblasts in a third culture medium. In embodiments, the third culture medium comprises one or more (e.g., 2, 3, or all) of an insulin, ascorbic acid, oncostatin, and necrosulfonamide. In embodiments, the third culture medium comprises an insulin, ascorbic acid, oncostatin, and necrosulfonamide.

In embodiments, the third culture medium comprises an insulin, such as a human insulin. In embodiments, the insulin is from a recombinant source. In embodiments, the insulin is a recombinantly produced human insulin. In embodiments, the insulin is present in the third culture medium at a concentration of about 1 μg/mL to about 100 μg/mL, about 1 μg/mL to about 25 μg/mL, or about 5 μg/mL to about 15 μg/mL. In embodiments, the insulin is present at a concentration of about or more than about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 15 μg/mL, or 20 μg/mL. In embodiments, the insulin is present at a concentration of about or at least about 1 μg/mL. In embodiments, the insulin is present at a concentration of about or at least about 5 μg/mL. In embodiments, the insulin is present at a concentration of about or at least about 10 μg/mL.

In embodiments, the third culture medium comprises ascorbic acid. In embodiments, the ascorbic acid is present in the third culture medium at a concentration of about 1 μg/mL to about 100 μg/mL, about 10 μg/mL to about 100 μg/mL, or about 30 μg/mL to about 70 μg/mL. In embodiments, the ascorbic acid is present at a concentration of about or more than about 10 μg/mL, 20 μg/mL, 30 μg/mL, 40 μg/mL, 50 μg/mL, 60 μg/mL, 70 μg/mL, 80 μg/mL, 90 μg/mL or 100 μg/mL. In embodiments, the ascorbic acid is present at a concentration of about or at least about 10 μg/mL. In embodiments, the ascorbic acid is present at a concentration of about or at least about 30 μg/mL. In embodiments, the ascorbic acid is present at a concentration of about or at least about 50 μg/mL.

In embodiments, the third culture medium comprises an oncostatin, such as a human oncostatin. In embodiments, the oncostatin is from a recombinant source. In embodiments, the oncostatin is a recombinantly produced human oncostatin. In embodiments, the oncostatin is present in the third culture medium at a concentration of about 1 ng/mL to about 100 ng/mL, about 1 ng/mL to about 50 ng/mL, or about 10 ng/mL to about 30 ng/mL. In embodiments, the oncostatin is present at a concentration of about or more than about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, or 50 ng/mL. In embodiments, the oncostatin is present at a concentration of about or at least about 5 ng/mL. In embodiments, the oncostatin is present at a concentration of about or at least about 10 ng/mL. In embodiments, the oncostatin is present at a concentration of about or at least about 20 ng/mL.

In embodiments, the third culture medium comprises necrosulfonamide. In embodiments, the necrosulfonamide is present in the third culture medium at a concentration of about 1 nM to about 100 nM, about 10 nM to about 100 nM, or about 30 nM to about 70 nM. In embodiments, the necrosulfonamide is present at a concentration of about or more than about 10 nM, 20 nM, 30 nM, 40 nM, 50 nM, 60 nM, 70 nM, 80 nM, 90 nM or 100 nM. In embodiments, the necrosulfonamide is present at a concentration of about or at least about 10 nM. In embodiments, the necrosulfonamide is present at a concentration of about or at least about 30 nM. In embodiments, the necrosulfonamide is present at a concentration of about or at least about 50 nM.

In embodiments, the third culture medium includes (a) the insulin at a concentration of about 1 μg/mL to about 25 μg/mL; (b) the ascorbic acid at a concentration of about 10 μg/mL to about 100 μg/mL; (c) the oncostatin at a concentration of about 1 ng/mL to about 100 ng/mL; and/or (d) the necrosulfonamide at a concentration of about 10 nM to about 100 nM.

In embodiments, the myoblasts are cultured in the third culture medium for about 1 to about 12 days, about 1 to about 10 days, or about 6 to about 8 days. In embodiments, the myoblasts are cultured in the third culture medium for about or more than about 1 day, 2 days, 3 days, 4 days, 5 days, 6 days, 7 days, 8 days, 9 days, or 10 days. In embodiments, the myoblasts are culture in the third culture medium for about or at least about 6 days. In embodiments, the myoblasts are culture in the third culture medium for about or at least about 7 days. In embodiments, the myoblasts are cultured in the third culture medium for about or at least about 8 days. In embodiments, the myoblasts are cultured in the third culture medium for a duration sufficient to form myotubes. In embodiments, the myotubes are characterized by (a) multiple nuclei, and (b) expression of one, two, or all of myosin heavy chain, myogenin, and dystrophin.

In embodiments, the method further comprises replacing the third culture medium with the second culture medium described herein. The culture medium can be replaced before and/or after formation of myotubes. In embodiments, culturing in the second culture medium is for a brief period (also referred to herein as a “pulse”), after which the second culture medium is replaced with the third culture medium. In embodiments, a pulse is for a duration of about 1 to about 24 hours, or about 4 to about 12 hours. In embodiments, a pulse is for a duration of about or less than about 24 hours, 20 hours, 18 hours, 16 hours, 14 hours, 12 hours, 10 hours, 8 hours, 6 hours, or 4 hours. In embodiments, a pulse is for a duration of about or less than about 18 hours. In embodiments, a pulse is for a duration of about or less than about 12 hours. In embodiments, a pulse is for a duration of about or less than about 8 hours. In embodiments, a first pulse is initiated after a period of about 1 to about 8 days, or about 3 to about 4 days of culturing in the third culture medium. In embodiments, the first pulse is initiated after a period of about 1 day, 2 days, 3 days, 4 days, 5 days, 6 days, 7 days, or 8 days of culturing in the third culture medium. In embodiments, the first pulse is initiated after a period of about 1 day. In embodiments, the first pulse is initiated after a period of about 3 days. In embodiments, the first pulse is initiated after a period of about 4 days. In embodiments, the first pulse is initiated after the formation of myotubes.

In embodiments, culturing the myoblasts to form myotubes comprises a plurality of cycles, wherein in cycle comprises a first step of culturing in the third culture medium followed by a second step (also referred to as a “pulse”) of culturing in the second culture medium. In embodiments, the plurality of cycles comprises 2, 3, 4, 5, 6, 7, 8, or more cycles. In embodiments, the first step comprises culturing in the third culture medium for about 1 to about 8 days, or about 3 to about 4 days of culturing in the third culture medium. In embodiments, the first step comprises culturing in the third culture medium for about 1 day, 2 days, 3 days, 4 days, 5 days, 6 days, 7 days, or 8 days. In embodiments, the first step comprises culturing in the third culture medium for about 3 to about 4 days. In embodiments, the second step comprises culturing in the second culture medium for about 1 to about 24 hours, or about 4 to about 12 hours. In embodiments, the second step comprises culturing in the second culture medium for about or less than about 24 hours, 20 hours, 18 hours, 16 hours, 14 hours, 12 hours, 10 hours, 8 hours, 6 hours, or 4 hours. In embodiments, the second step comprises culturing in the second culture medium for about 4 to about 12 hours. In embodiments, myotubes cultured according to the cycling procedure are maintained for about or more than about 10, 11, 12, 13, 14, 15, 20, 25, or more days. In embodiments, myotubes cultured according to the cycling procedure are maintained for about or more than about 10 days. In embodiments, myotubes cultured according to the cycling procedure are maintained for about or more than about 15 days. In embodiments, myotubes cultured according to the cycling procedure are maintained for about or more than about 21 days.

### Methods for Treatment

In some aspects, the present disclosure provides methods of treating subjects with cells produced according to methods disclosed herein, such as myoblast precursor cells, myoblasts, and/or myotubes. In embodiments, the methods comprise administering the cells to a subject in need thereof, or the use of such cells for treating a condition of the subject. In embodiments, the cells are used for treating muscular degenerative diseases or muscular disorders stemming from a variety of causes, including but not limited to genetic disorders, sporadic diseases, cachexia, muscle strain, muscle injury, muscle atrophy and/or muscle wasting as exemplified by different forms of cachexia, as well as sarcopenia and the general aging process. Cells provided herein may be used in cell therapies for such subjects, particularly therapies to replenish or supplement a subject's naturally-occurring skeletal muscle cells.

Subjects who may generally benefit from the cells and methods provided herein are subjects with a muscular disease or disorder that affects muscle function, tone or physiology. In some cases, the subjects may have a genetic disease (e.g., Huntington's disease, muscular dystrophy); in some cases, the subjects may have an acquired disorder (e.g., muscle atrophy caused by inactivity). Additionally, subjects with muscular dystrophy may have multi-system disorders with manifestations in body systems including the heart, gastrointestinal system, nervous system, endocrine glands, eyes and brain. Subjects in need of treatment can include those who have undergone muscle strain or injury. The muscle injury may be the result of a traumatic event, such as a slip or fall during an activity such exercise. Exemplary diseases or disorders that may be exhibited by the subjects treated using the methods disclosed herein include: muscular dystrophy, Huntington's disease, Merosin deficiency 1A, nemaline myopathy, and Spinal Muscular Atrophy (SMA). Examples of muscular dystrophies that may be treated or improved by the disclosed cells include Becker, congenital, facioscapulohumeral (FSH), myotonic (type I and II), oculopharyngeal, distal, Duchenne muscular dystrophy, and Emery-Dreifuss muscular dystrophy. Duchenne and Becker muscular dystrophies are caused by a mutation of a gene located on the X chromosome and predominantly affect males, although females can sometimes have severe symptoms as well. Subjects in need of treatment may also include subjects experiencing muscle atrophy or wasting, including muscle atrophy that may occur as a result of cachexia or wasting syndrome. Cachexia may be accompanied by muscle atrophy, loss of weight, fatigue, weakness, and significant loss of weight. The methods of treatment provided herein may help reverse some of these symptoms, particularly muscle atrophy and weakness. Subjects with cachexia may include patients with cancer, acquired immune deficiency syndrome (AIDS), chronic obstructive lung disease, multiple sclerosis, congestive heart failure, tuberculosis, familial amyloid polyneuropathy, gadolinium poisoning, mercury poisoning (acrodynia) and hormonal deficiency.

In embodiments, the myoblast precursor cells, myoblasts, or myotubes are prepared for administration with a pharmaceutically acceptable excipient. In general, “pharmaceutically acceptable excipient” and “pharmaceutically acceptable carrier” refer to a substance that aids the administration of the cells to and engraftment in a subject, and can be included in compositions of the present disclosure without causing a significant adverse toxicological effect on the subject. Non-limiting examples of pharmaceutically acceptable excipients include water, NaCl, normal saline solutions, lactated Ringer's, normal sucrose, normal glucose, binders, fillers, lubricants, salt solutions (such as Ringer's solution), oils, gelatins, carbohydrates such as lactose, amylose or starch, fatty acid esters. Such preparations can be sterilized and, if desired, mixed with auxiliary agents such as lubricants, salts for influencing osmotic pressure, buffers, and the like that do not deleteriously react with the cells of the disclosure. In embodiments, the cells are administered within an extracellular matrix (ECM). One of skill in the art will recognize that other pharmaceutical excipients are useful in the present disclosure.

### Culture Systems and Compositions

In some aspects, the present disclosure provides culture systems and compositions for culturing pluripotent cells, myoblast precursors, myoblasts, and myotubes, as disclosed herein. In embodiments, compositions include one or more media as disclosed herein, such as with regard to any of the various aspects and methods disclosed herein. The media (e.g., the culture medium for differentiating pluripotent cells, the second culture media, or the third culture media) may comprise any of the components or combinations thereof as described herein, such as with regarding to the methods described above. In some aspects, the present disclosure provides kits and culture systems comprising one or more of the culture media or components thereof, as described herein.

In some aspects, the present disclosure provides a culture medium for differentiating pluripotent cells. In embodiments, the culture medium is a culture medium as disclosed above, with respect to methods for differentiating pluripotent cells. In embodiments, the medium comprises a fetal bovine serum (FBS), a chick embryo extract, and a fetuin. In embodiments, (a) the FBS is present at a concentration of about 1% to about 10%, (b) the chick embryo extract is present at a concentration of about 0.1% to about 5%, and/or (c) the fetuin is present at a concentration of about 10 ng/mL to about 100 ng/mL. In embodiments, the culture medium further includes one or more of (e.g., 2, 3, 4, 5, or all of) an epidermal growth factor (EGF), an insulin, dexamethasone, a ROCK inhibitor, ascorbic acid, a basic fibroblast growth factor (bFGF), a GSK3 inhibitor, or an ALK5 inhibitor. In embodiments, (a) the EGF is a human EGF, (b) the insulin is a human insulin, (c) the ROCK inhibitor is Y-27632, (d) the bFGF is a human bFGF, (e) the GSK3 inhibitor is CHIR99021, and/or (f) the ALK5 inhibitor is RepSox. In embodiments, the culture medium further includes pluripotent cells or myoblast precursor cells. In embodiments, the culture medium does not include a horse serum.

In some aspects, the invention provides a culture system for differentiating pluripotent cells. In embodiments, the culture system comprises a first culture medium, a second culture medium, and a third culture medium. In embodiments, the first culture medium is a culture medium for differentiating pluripotent cells into myoblast precursor cells, as disclosed herein. In embodiments, the second culture medium is a culture medium for differentiating myoblast precursor cells into myoblasts, as disclosed herein. In embodiments, the third culture medium is a medium for culturing myoblasts to form myotubes, as disclosed herein. In embodiments, the culture system comprises: (a) a first culture medium including one or more of (and preferably all of) a fetal bovine serum (FBS), a chick embryo extract, and a fetuin; (b) a second culture medium including one or more of (e.g., 2, 3, 4, 5, or all of) a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF); and (c) a third culture medium including one or more of (e.g., 2, 3, or 4 of) an insulin, ascorbic acid, oncostatin, and necrosulfonamide.

In embodiments, the first culture medium includes (a) the FBS at a concentration of about 1% to about 10%, (b) the chick embryo extract at a concentration of about 0.1% to about 5%, and/or (c) the fetuin at a concentration of about 10 ng/mL to about 100 ng/mL. In embodiments, the first culture medium further includes one or more of an epidermal growth factor (EGF), an insulin, dexamethasone, a ROCK inhibitor, ascorbic acid, a basic fibroblast growth factor (bFGF), a GSK3 inhibitor, or an ALK5 inhibitor.

In embodiments, the second culture medium includes the horse serum, the fetuin, the EGF, the insulin, dexamethasone, the ROCK inhibitor, ascorbic acid, the ALK5 inhibitor, a hepatocyte growth factor (HGF), the bFGF, the insulin-like growth factor-1 (IGF-1), oncostatin, and the platelet-derived growth factor (PDGF).

In embodiments, the third culture medium includes the insulin, ascorbic acid, oncostatin, and necrosulfonamide. In embodiments, the third culture medium includes (a) the insulin at a concentration of about 1 μg/mL to about 25 μg/mL; (b) the ascorbic acid at a concentration of about 10 μg/mL to about 100 μg/mL; (c) the oncostatin at a concentration of about 1 ng/mL to about 100 ng/mL; and/or (d) the necrosulfonamide at a concentration of about 10 nM to about 100 nM.

In embodiments, the first culture medium includes pluripotent cells or myoblast precursor cells.

In embodiments, the second culture medium includes myoblast precursor cells or myoblasts.

In embodiments, the third culture medium includes myoblasts or myotubes.

### Cells and Compositions

In some aspects, the present disclosure provides cells and compositions produced according to methods disclosed herein, such as myoblast precursor cells, myoblasts, myotubes, and compositions derived therefrom. In some aspects, the present disclosure provides myotubes produced according to methods as disclosed herein, including embodiments thereof. In embodiments, the myoblasts and myotubes produced according to the methods disclosed herein differ from their natural counterparts as a result of processes for forming iPSCs or differentiating cells therefrom. For example, populations of iPSC-derived cells may exhibit an increased average telomere length than their naturally-occurring counterparts. In embodiments, the telomeres are increased about or at least about 5%, 10%, 25%, or more, as compared to a naturally occurring counterpart cell.

In some aspects, the present disclosure provides a pharmaceutical composition including the myoblast precursor cells, myoblasts, or myotubes as disclosed herein, including embodiments thereof, and a pharmaceutically acceptable carrier. In some aspects, the present disclosure provides a use of the cells disclosed herein in the production of a pharmaceutical composition. Pharmaceutically acceptable carriers for tissue to be used for transplantation are known. In embodiments, the myotubes are maintained in a culture medium disclosed herein, and are washed (e.g., with a sterile saline solution) prior to transplantation. In embodiments, the myotubes are prepared for transplanting to a subject in need thereof. In embodiments, the subject suffers from a musculoskeletal condition or injury. In embodiments, the musculoskeletal condition is a condition that causes or results in muscle atrophy. Muscle atrophy can result from treatment with a glucocorticoid such as Cortisol, dexamethasone, betamethasone, prednisone, methylprednisolone or prednisolone. Muscle atrophy can also be a result of denervation due to nerve trauma or a result of degenerative, metabolic or inflammatory neuropathy. For example, muscle atrophy can be a result of an adult motor neuron disease, Guillain-Barre syndrome, infantile spinal muscular atrophy, amyotrophic lateral sclerosis, juvenile spinal muscular atrophy, autoimmune motor neuropathy with multifocal conductor block, paralysis due to stroke or spinal cord injury, skeletal immobilization due to trauma, prolonged bed rest, voluntary inactivity, involuntary inactivity, and metabolic stress or nutritional insufficiency. Muscle atrophy can be a result of myopathy, including for example myotonia, a congenital myopathy, including nemalene myopathy, multi/minicore myopathy and myotubular (centronuclear) myopathy; mitochondrial myopathy; familial periodic paralysis; inflammatory myopathy; metabolic myopathy, such as caused by a glycogen or lipid storage disease; dermatomyositis; polymyositis; inclusion body myositis; myositis ossificans; rhabdomyolysis and myoglobinurias. Myopathy may be caused by a muscular dystrophy syndrome, such as Duchenne muscular dystrophy (DMD), Becker muscular dystrophy (also known as benign pseudohypertrophic muscular dystrophy), myotonic dystrophy, scapulohumeral and fascioscapulohumeral muscular dystrophy, Emery-Dreifuss muscular dystrophy, oculopharyngeal muscular dystrophy, limb girdle muscular dystrophy, Fukuyama congenital muscular dystrophy, or hereditary distal myopathy. In embodiments, the subject has a muscle injury, such as a tear, laceration, or perforation of one or more muscles. In embodiments, the pharmaceutically acceptable carrier comprises an extracellular matrix to facilitate engraftment. In general, an extracellular matrix a matrix comprises a web of molecules located outside or external to cells that regulate cell-cell communication, cell signaling, cell adhesion, spacing, location and/or orientation, although without limitation thereto. The molecular components of ECM may include proteoglycans, heparan sulphate, chondroitin sulphate, keratin, collagens (e.g types I-XIV), elastins, laminin and fibronectin, although without limitation thereto. In some embodiments, the ECM may be present in the form of MATRIGEL™. In embodiments, the myotubes are provided in an amount effective to treat the condition or injury of the subject.

In some aspects, the present disclosure provides a use of the myotubes as disclosed herein, including embodiments thereof, in the production of an edible composition. In general, the edible composition comprises myoblasts or myotubes produced according to a method disclosed herein, in whole or in part (e.g., as in an extract), as a component of a food product. The myoblasts and myotubes of the present disclosure may be used in the preparation of any of a variety of food products, where they can contribute to the taste, texture and nutritional content. The synthetic food products of the present disclosure may be pickled, boiled, cooked, smoked, fried, baked, dried or frozen, and may be prepared as a snack or as part of a meal. The final food (edible) products may be configured in any of a variety of consumption forms including, but not limited to, soup, puree, paste, pie, pellets, crumbles, gel, powder, granules, tablet, chips, capsule, spread, sausage, and the like. The final food product can be prepared on a 3D printer. Examples of 3D printing of food includes those developed by NOVAMEAT, JET-EAT, MEATECH and other companies.

In embodiments, the food product comprises one or more flavorants. The flavorant may be added during the mixing step, or may be mixed with any of the components (e.g., the cultured cells) before the mixing step. Examples of taste and sensation producing flavorants include artificial sweeteners, glutamic acid salts, glycine salts, guanylic acid salts, inosinic acid salts, ribonucleotide salts, and organic acids, including acetic acid, citric acid, malic acid, tartaric acid, and polyphenolics. A few representative examples of common flavor aromatics include isoamyl acetate (banana), cinnamic aldehyde (cinnamon), ethyl propionate (fruity), limonene (orange), ethyl-(E,Z)-2,4-decadienoate (pear), allyl hexanoate (pineapple), ethyl maltol (sugar, cotton candy), methyl salicylate (wintergreen), and mixtures thereof.

In embodiments, the food product comprises a color enhancer (colorant) which may be added to the cultured cells for making the food product visually more attractive. Additionally, the colorant may function as a physiological antioxidant, thus providing another essential nutrient. For example, colored antioxidants such as some flavonoids, carotenoids, anthocyanins and the like, from tomatoes, black currants, grapes, blueberries, cranberries and the like may be used. In embodiments, the colorant is a natural product or the refined or partially refined product. For example, refined catechins, resveratrol, anthocyanin, beta-carotenes, lycopene, lutein, zeaxanthin and the like may be used as the colorant.

### Illustrative Embodiments

The present disclosure provides the following illustrative embodiments.

Embodiment P1: A method of differentiating pluripotent cells, the method comprising culturing pluripotent cells in a culture medium comprising one or more of a fetal bovine serum (FBS), a chick embryo extract, and a fetuin; wherein the differentiation produces myoblast precursor cells.

Embodiment P2: The method of Embodiment P1, wherein the FBS is a charcoal-stripped FBS, and/or the fetuin is a bovine fetuin.

Embodiment P3: The method of any one of Embodiments P1 or P2, wherein the culture medium comprises the FBS, the chick embryo extract, and the fetuin.

Embodiment P4: The method of any one of Embodiments P1-P3, wherein (a) the FBS is present at a concentration of about 1% to about 10%; (b) the chick embryo extract is present at a concentration of about 0.1% to about 5%; and/or (c) the fetuin is present at a concentration of about 1 mg/mL to about 100 mg/mL.

Embodiment P5: The method of any one of Embodiments P1-P4, wherein the culture medium further comprises one or more of an epidermal growth factor (EGF), an insulin, dexamethasone, a ROCK inhibitor, ascorbic acid, a basic fibroblast growth factor (bFGF), a GSK3 inhibitor, or an ALK5 inhibitor.

Embodiment P6: The method of Embodiment P5, wherein (a) the EGF is a human EGF, (b) the insulin is a human insulin, (c) the ROCK inhibitor is Y-27632, (d) the bFGF is a human bFGF, (e) the GSK3 inhibitor is CHIR99021, and/or (f) the ALK5 inhibitor is RepSox.

Embodiment P7: The method of any one of Embodiments P1-P6, wherein the pluripotent cells are induced pluripotent stem cells (iPSCs).

Embodiment P8: The method of any one of Embodiments P1-P7, wherein the pluripotent cells are cultured in the culture medium for about 5 to about 15 days.

Embodiment P9: The method of any one of Embodiments P1-P8, wherein the myoblast precursor cells comprise satellite-like cells characterized by expression of one or more of Pax3, Pax7, CD56/NCAM, Nanog, POU5F1/Oct-4, and SSEA-4.

Embodiment P10: The method of any one of Embodiments P1-P9, wherein the culture medium does not comprise a horse serum.

Embodiment P11: The method of any one of Embodiments P1-P10, wherein the pluripotent cells are cultured on a surface pretreated with collagen I.

Embodiment P12: The method of any one of Embodiments P1-P11, further comprising culturing the myoblast precursor cells in a second culture medium, wherein the second culture medium comprises one or more of a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF); wherein the culturing in the second culture medium produces myoblasts.

Embodiment P13: The method of Embodiment P12, wherein the myoblast precursor cells are cultured in the second culture medium for about 3 to about 14 days.

Embodiment P14: The method of Embodiment P12 or P13, wherein the myoblasts are characterized by expression of one or both of MyoD and Desmin.

Embodiment P15: The method of any one of Embodiments P12-P14, further comprising culturing the myoblasts in a third culture medium, wherein the third culture medium comprises one or more of an insulin, ascorbic acid, oncostatin, and necrosulfonamide; wherein the culturing in the third culture medium produces myotubes.

Embodiment P16: The method of Embodiment P15, wherein the insulin in the third culture medium is a human insulin.

Embodiment P17: The method of Embodiment P15 or P16, wherein the third culture medium comprises the insulin, ascorbic acid, oncostatin, and necrosulfonamide.

Embodiment P18: The method of any one of Embodiments P15-P17, wherein the third culture medium comprises (a) the insulin at a concentration of about 1 μg/mL to about 25 μg/mL; (b) the ascorbic acid at a concentration of about 10 μg/mL to about 100 μg/mL; (c) the oncostatin at a concentration of about 1 ng/mL to about 100 ng/mL; and/or (d) the necrosulfonamide at a concentration of about 10 nM to about 100 nM.

Embodiment P19: The method of any one of Embodiments P15-P18, wherein the myoblasts are cultured in the third culture medium for about 1 to about 10 days.

Embodiment P20: The method of any one of Embodiments P15-P19, further comprising culturing the myotubes in the second culture medium.

Embodiment P21: The method of Embodiment P20, wherein the myotubes are cultured in the second culture medium for about 1 to about 24 hours.

Embodiment P22: The method of Embodiment P20 or P21, wherein culturing the myotubes comprises a plurality of cycles, each cycle comprising (a) culturing the myotubes in the third culture medium, and (b) culturing the myotubes in the second culture medium.

Embodiment P23: The method of Embodiment P22, wherein the myotubes are maintained in culture for more than 10 days.

Embodiment P24: The method of any one of Embodiments P15-P23, wherein the myotubes are characterized by (a) multiple nuclei, and (b) expression of one or more of myosin heavy chain, myogenin, and dystrophin.

Embodiment P25: Myotubes produced according to a method of any one of Embodiments P15-P24.

Embodiment P26: A pharmaceutical composition comprising the myotubes of Embodiment P25 and a pharmaceutically acceptable carrier.

Embodiment P27: Use of the myotubes of Embodiment P25 in the production of a pharmaceutical composition.

Embodiment P28: Use of the myotubes of Embodiment P25 in the production of an edible composition.

Embodiment P29: A culture medium for differentiating pluripotent cells, the medium comprising a fetal bovine serum (FBS), a chick embryo extract, and a fetuin.

Embodiment P30: The culture medium of Embodiment P29, wherein (a) the FBS is present at a concentration of about 1% to about 10%, (b) the chick embryo extract is present at a concentration of about 0.1% to about 5%, and/or (c) the fetuin is present at a concentration of about 1 mg/mL to about 100 mg/mL.

Embodiment P31: The culture medium of Embodiment P29 or P30, further comprising one or more of an epidermal growth factor (EGF), an insulin, dexamethasone, a ROCK inhibitor, ascorbic acid, a basic fibroblast growth factor (bFGF), a GSK3 inhibitor, or an ALK5 inhibitor.

Embodiment P32: The culture medium of Embodiment P31, wherein (a) the EGF is a human EGF, (b) the insulin is a human insulin, (c) the ROCK inhibitor is Y-27632, (d) the bFGF is a human bFGF, (e) the GSK3 inhibitor is CHIR99021, and/or (f) the ALK5 inhibitor is RepSox.

Embodiment P33: The culture medium of any one of Embodiments P29-P32, further comprising pluripotent cells or myoblast precursor cells.

Embodiment P34: The culture medium of any one of Embodiments P29-P33, wherein the culture medium does not comprise a horse serum.

Embodiment P35: A culture system for differentiating pluripotent cells, the culture system comprising: (a) a first culture medium comprising one or more of a fetal bovine serum (FBS), a chick embryo extract, and a fetuin; (b) a second culture medium comprising one or more of a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF); and (c) a third culture medium comprising one or more of an insulin, ascorbic acid, oncostatin, and necrosulfonamide.

Embodiment P36: The culture system of Embodiment P35, wherein the first culture medium comprises the FBS, the chick embryo extract, and the fetuin.

Embodiment P37: The culture system of Embodiment P35 or P36, wherein the first culture medium comprises (a) the FBS at a concentration of about 1% to about 10%, (b) the chick embryo extract at a concentration of about 0.1% to about 5%, and/or (c) the fetuin at a concentration of about 1 mg/mL to about 100 mg/mL.

Embodiment P38: The culture system of any one of Embodiments P35-P37, wherein the first culture medium further comprises one or more of an epidermal growth factor (EGF), an insulin, dexamethasone, a ROCK inhibitor, ascorbic acid, a basic fibroblast growth factor (bFGF), a GSK3 inhibitor, or an ALK5 inhibitor.

Embodiment P39: The culture system of any one of Embodiments P35-P38, wherein the second culture medium comprises the horse serum, the fetuin, the EGF, the insulin, dexamethasone, the ROCK inhibitor, ascorbic acid, the ALK5 inhibitor, the hepatocyte growth factor (HGF), the bFGF, the insulin-like growth factor-1 (IGF-1), oncostatin, and the platelet-derived growth factor (PDGF).

Embodiment P40: The culture system of any one of Embodiments P35-P39, wherein the third culture medium comprises the insulin, ascorbic acid, oncostatin, and necrosulfonamide.

Embodiment P41: The culture system of any one of Embodiments P35-P40, wherein the third culture medium comprises (a) the insulin at a concentration of about 1 μg/mL to about 25 μg/mL; (b) the ascorbic acid at a concentration of about 10 μg/mL to about 100 μg/mL; (c) the oncostatin at a concentration of about 1 ng/mL to about 100 ng/mL; and/or (d) the necrosulfonamide at a concentration of about 10 nM to about 100 nM.

Embodiment P42: The culture system of any one of Embodiments P35-P41, wherein the first culture medium comprises pluripotent cells or myoblast precursor cells.

Embodiment P43: The culture system of any one of Embodiments P35-P42, wherein the second culture medium comprises myoblast precursor cells or myoblasts.

Embodiment P44: The culture system of any one of Embodiments P35-P43, wherein the third culture medium comprises myoblasts or myotubes.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1: Directed Differentiation of iPSCs to Skeletal Muscle (SKM) Cells

Basal media, complete MCDB media (cMCDB), was prepared as follows. 10.8 g of MCDB powder (MYBIOSOURCE, Cat. #MBS652968) was dissolved in 950 mL of ddH₂O until completely solubilized. The following components were then added: 16 mL of bicarbonate (7.5%), 250 μL of 1N HCl to adjust pH to between 7.5 to 7.7, and ddH₂O to bring the solution to 1 L. The solution was filtered using a 0.22 micron membrane filter, and stored at 4° C. for up to two months. Medium may be frozen for longer storage.

The basal medium was supplemented according to Tables 1, 2, or 3 to prepare the first culture medium, second culture medium, or third culture medium, respectively.

TABLE 1
SKM Cell differentiation Stage 1 medium:

| CHEMICAL | Stock Conc. | | Final Conc. | | Amount/1 L | |
|---|---|---|---|---|---|---|
| cMCDB Base Media | — | | | | | |
| (complete) | | | | | | |
| CS-FBS | — | | 5% | | 50 | ml |
| CEE | — | | 0.5% | | 5 | ml |
| Fetuin (Bovine) | 50 | mg/mL | 50 | ug/mL | 1 | ml |
| hr-EGF | 20 | ug/mL | 10 | ng/mL | 0.5 | ml |
| Insulin (Human) | 10 | mg/mL | 10 | ug/ml | 1 | ml |
| Dexamethasone | 1 | mg/mL | 0.4 | ug/mL | 0.4 | ml |
| Rock Inhibitor | 10 | mM | 10 | uM | 1 | ml |
| (Y-27632) | | | | | | |
| Ascorbic Acid | 100 | mg/mL | 50 | ug/mL | 0.5 | ml |
| (Vitamin C) | | | | | | |
| hr-bFGF | 100 | ug/mL | 1 | ng/mL | 10 | ul |

| | | | | | |
|---|---|---|---|---|---|
| CHIR99021 | 20 mM (9.302 | 3 | uM | 150 | ul |
| | mg/mL) | | | | |
| Alk5 Inhibitor | 10 mM (2.873 | 2 | uM | 200 | ul |
| (RepSox) | mg/mL) | | | | |

TABLE 2
SKM Cell Differentiation Stage 2 Medium
(Myoblast expansion medium)

| CHEMICAL | Stock Conc. | | Final Conc. | | Amount/1 L | |
|---|---|---|---|---|---|---|
| cMCDB Base Media | — | | 94.27% | | | |
| (complete) | | | | | | |
| Horse Serum | — | | 5% | | 50 | ml |
| Fetuin (Bovine) | 50 | mg/mL | 50 | ug/mL | 1 | ml |
| hr-EGF | 20 | ug/mL | 10 | ng/mL | 0.5 | ml |
| Insulin (Human) | 10 | mg/mL | 10 | ug/ml | 1 | ml |
| Dexamethasone | 1 | mg/mL | 0.4 | ug/mL | 400 | ul |
| Rock Inhibitor | 10 | mM | 10 | uM | 1 | ml |
| (Y-27632) | | | | | | |
| Ascorbic Acid | 100 | mg/mL | 50 | ug/mL | 0.5 | ml |
| (Vitamin C) | | | | | | |
| SB431542 | 10 mM (3.846 | | 2 | uM | 200 | ul |
| | mg/mL) | | | | | |
| HGF | 20 | ug/mL | 20 | ng/mL | 1 | ml |
| hr-bFGF | 100 | ug/mL | 20 | ng/mL | 200 | ul |
| IGF1 | 50 | ug/ml | 10 | ng/mL | 200 | ul |
| Oncostatin | 20 | ug/mL | 10 | ng/mL | 0.5 | ml |
| PDGF | 10 | ug/mL | 10 | ng/mL | 1 | ml |

TABLE 3
SKM Cell Differentiation Stage 3 Medium
(Myotube differentiation medium)

| CHEMICAL | Stock Conc. | | Final Conc. | | Amount/1 L | |
|---|---|---|---|---|---|---|
| cMCDB Base Media | — | | | | | |
| (complete) | | | | | | |
| Insulin (Human) | 10 | mg/mL | 10 | ug/ml | 1 | ml |
| Ascorbic Acid | 100 | mg/mL | 50 | ug/mL | 0.5 | ml |
| (Vitamin C) | | | | | | |
| Oncostatin | 20 | ug/mL | 10 | ng/mL | 0.5 | ml |

| | | | | | |
|---|---|---|---|---|---|
| Necrosulfonamide | 10 mM (4.6147 | 50 | nM | 5 | ul |
| | mg/ml) | | | | |

Differentiation from iPSCs to Satellite-Like/Myogenic Precursor Cells (Stage 1):

iPS cells were maintained in normal mTesR media for 2-3 passages before starting differentiation. Before staring, frozen Stage I medium was thawed at 4° C. before use. The medium may be stored at 4° C. for up to two weeks. Coated plates with collagen I were prepared by adding the appropriate amount of collagen I into 0.01N HCl as described in Table 4 and the protein was allowed to bind to the plates for at least 2 hours at 37° C., or at room temperature (RT) overnight. After this binding step, the excess fluid was removed from the coated surfaces and the plates allowed to dry overnight in a ventilated hood. Once dried, the plates were rinsed with sterile tissue culture grade water or PBS, before introducing the cells and culture medium.

TABLE 4
Amounts of Collagen I Added by Vessel Size

| | Growth | | 0.01N | Collagen I |
|---|---|---|---|---|
| | area | Collagen I | HCl | Volume/well |
| Vessel name | (cm²) | (ug/well) | (ml) | (ul) |
| 96-well plate | 0.32 | 3.2 | 0.02 | 1 |
| 48-well plate | 0.95 | 9.5 | 0.1 | 2.5 |
| 6-well plate | 9.5 | 95 | 1 | 25 |
| T75 flask | 75 | 750 | 5 | 200 |

At day 0, the cells were dissociated with Accutase and neutralized with Stage 1 medium (described in Table 1). The cells were passed through 70 μm mesh and seeded at 5,000 cells/cm² onto collagen 1-coated plates, one 6-well plate and one 48-well plates in 2 mL and 0.25 mL Stage I medium, respectively. One 6 well plate was used for the second stage of differentiation, while the 48-well plate was used for immunocytochemistry (ICC) at end of Stage I.

Stage 1 medium was changed every other day until about 100% confluent (which was reached in approximately 8-12 days). Cell morphology was monitored by imaging taken at Day 0, Day 5, Day 8, and Day 12, as needed. At the end of Stage I, in which cells were at about 100% confluence, 48-well plates were fixed with 4% PFA and subjected to ICC for Pax3, Pax7, CD56/NCAM, Nanog, POU5F1/Oct-4, SSEA-4. At this stage, cells from 6-well plates were collected by 0.05% Trypsin/EDTA and frozen down in CS10 frozen medium at 3-4 million cells per vial.

Ideally, more than 50% of the cells express Pax3 at the end of Stage I differentiation. Pax7 was also typically expressed at the end of differentiation, but it may be minimal. CD56 expression was preferably greater than 20% at the end of this phase of the differentiation. Cells at the end of differentiation only expressed low levels of pluripotency factors (e.g., Nanog, POU5F1/Oct-4, and SSEA-4) compared to high-level expression at the beginning.

Differentiation from Satellite-Like/Myogenic Precursor Cells to Myoblast (Stage 2):

The preferred vessels were coated with collagen I one day before seeding satellite-like cells from State 1. Frozen Stage 2 medium was thawed at 4° C. before use. The medium may be stored at 4° C. for up to two weeks. One vial of Satellite-like/myogenic precursor cells was thawed and seeded into collagen I-coated vessels at 5,000 cells/cm². In parallel, one 48-well collagen 1-coated plate was seeded in same density for immunocytochemistry and imaging.

The cells were then incubated in Stage 2 media for 6-9 days with the medium being changed every other day. The cell morphology was monitored by imaging taken at Day 1, Day 6, Day 7, Day 8 and Day 9 as needed. At end of Stage 2, in which the cells were about 100% confluent, the 48-well plate were fixed and analyzed for expression of MyoD and Desmin via immunocytochemistry. Ideally, more than 30% of cells express MyoD and more than 10% of cells express Desmin. The cells from the vessels were harvested by 0.05% Trypsin/EDTA and cryopreserved at 1-3 M/vials.

Formation of Myotubes from Myoblasts (Stage 3):

The preferred vessels were coated with collagen I one day before seeding myoblasts. The frozen Stage 3 medium was thawed at 4° C. before use. The medium may be stored at 4° C. for up to two weeks. One vial of frozen myoblasts was thawed and the myoblasts were seeded into one 48-well plate and appropriate vessels at 16,000-30,000 cells/cm² density in Stage 2 medium.

Cells were cultured for 3-4 days until about 100% confluent with Stage 2 medium change every other day. Then, the culture medium was changed to Stage 3 medium and morphology changes began to happen overnight.

Cells with appropriate elongated, multinucleated morphology could be observed anywhere from day 2-7 post Stage 3 medium change. The cells were then fixed when morphology looked best, which for most lines was at days 3-5. Once fixed, the cells were analyzed for expression of MHC, Myogenin, and Dystrophin.

Ideally, more than 75% of cells express MHC; more than 50% of cells express Dystrophin; and more than 30% of cells express Myogenin.

Depending upon the downstream application, the myotubes could be used in 2-7 days in Stage 3 medium which is preferably changed once every two days to maintain myotubes.

### Example 2: Comparison of Differentiation Protocols

### FBS and CEE Compared to Horse Serum Differentiation Medium

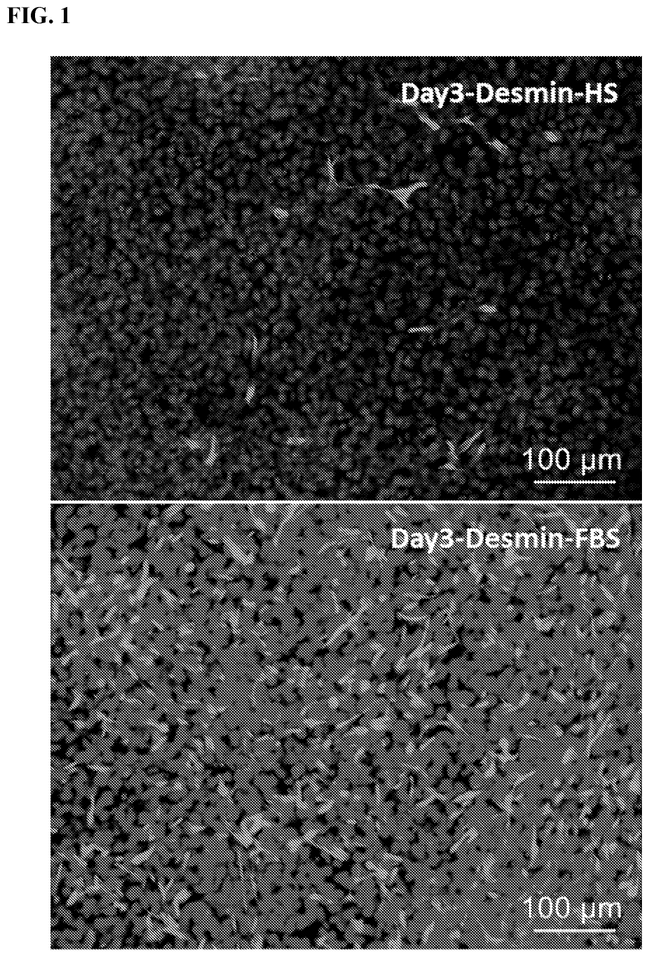

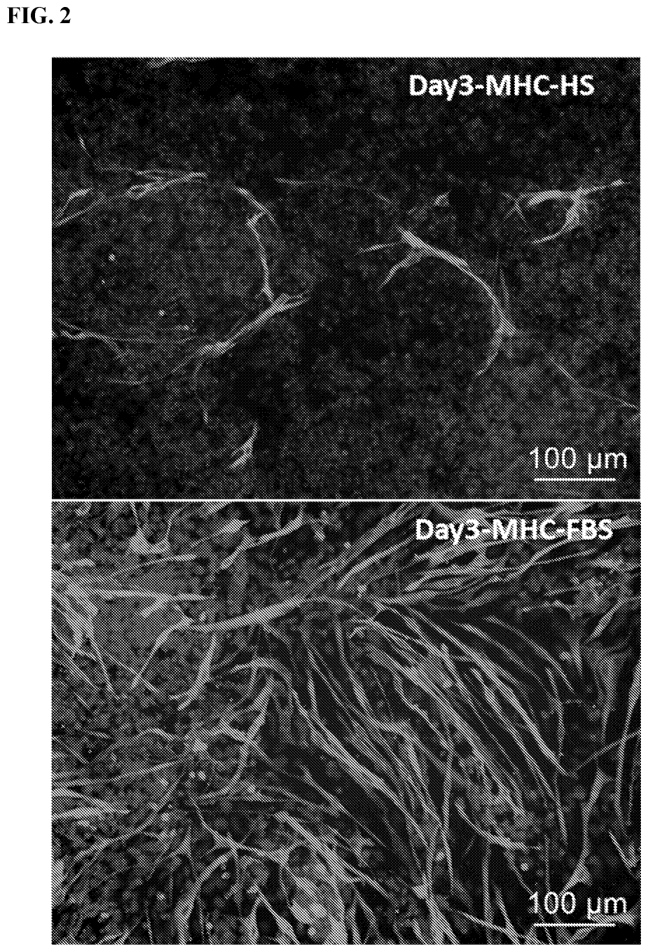

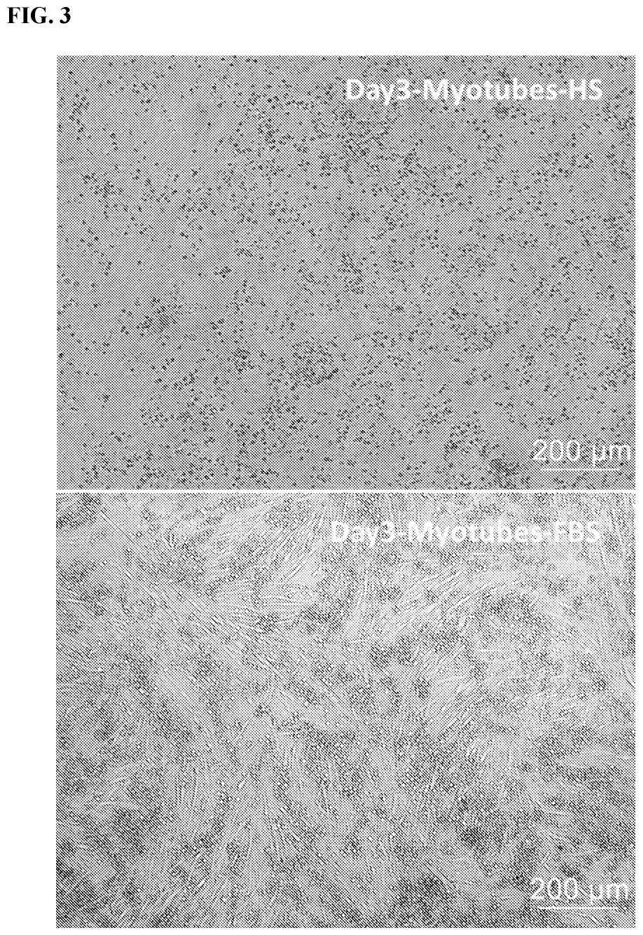

Satellite like progenitors were generated either with horse serum (HS) in the culture medium, or with FBS and chick embryo extract in the culture medium in accordance with Stage 1 differentiation described in Example 1. The satellite like progenitors were then used for myoblast production in accordance with Stage 2 differentiation described above. The myoblasts were seeded in a collagen I-coated plate at a density of 3.2×10⁴/cm² under myoblast expansion medium (Stage 2 medium). After three days, the cells were fixed and stained with Desmin (FIG. 1), or the Stage 2 medium was replaced with myotube differentiation medium (Stage III medium) and continued to culture for another three days before being fixed and stained for MHC (FIG. 2). FIGS. 1, 2 and 3 show a significant increase in the number of myoblast (FIG. 1) and myotubes (FIGS. 2 and 3) obtained by using FBS and CEE in Stage 1, as compared to horse serum. Moreover, Satellite-like cells using FBS Stage I medium grow faster than HS Stage I medium (8-10 days vs 10-13 days), representing a 20-30% reduction in differentiation time. Stage 1 cells with FBS media also attached more easily on Collagen coated vessels, leading to reduce experimental variation.

Effect of Freshly Coating Culture Plates with Collagen I

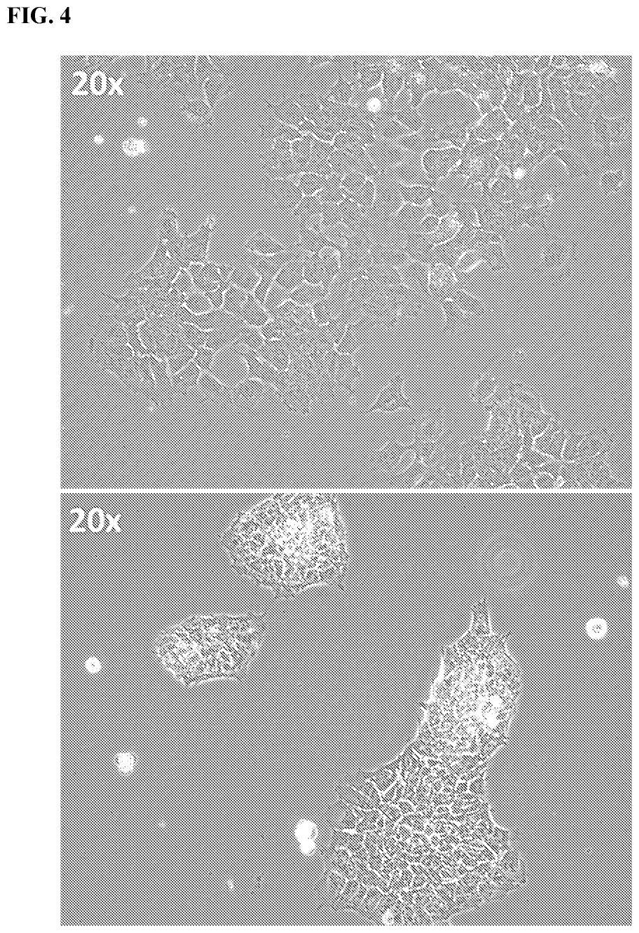

Single iPS cells were seeded in satellite-like differentiation medium (Stage 1 medium) at a density of about 5000 cell/cm² in either a 6-well plate freshly-coated collagen I or BIOCOAT plate purchased from purchased from CORNING© (Cat. #356400). As shown on FIG. 4, by day 4, cells in the freshly-coated plate (top panel) presented a more differentiated morphology, while cells in the BIOCOAT plate (bottom panel) presented a more clustered mESC-like morphology.

### Effect of Culture Medium “Pulsing” on Myotube Maintenance

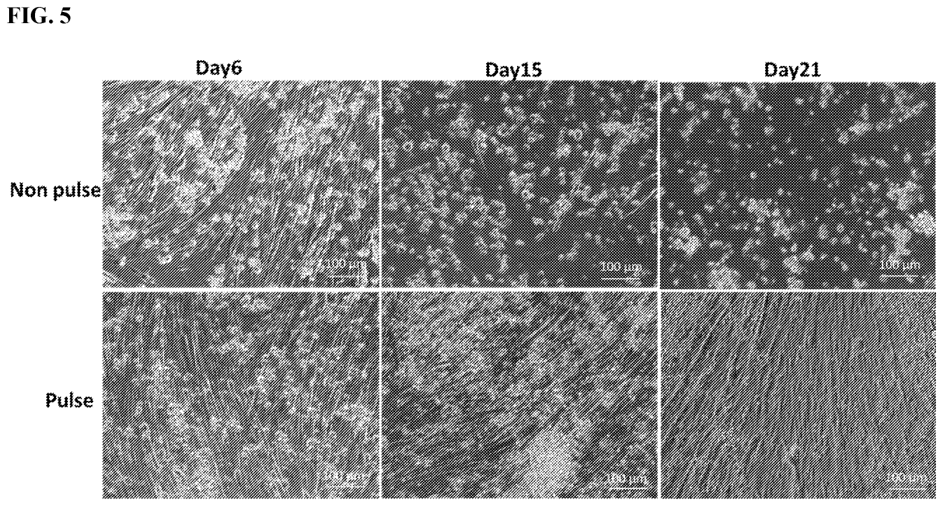

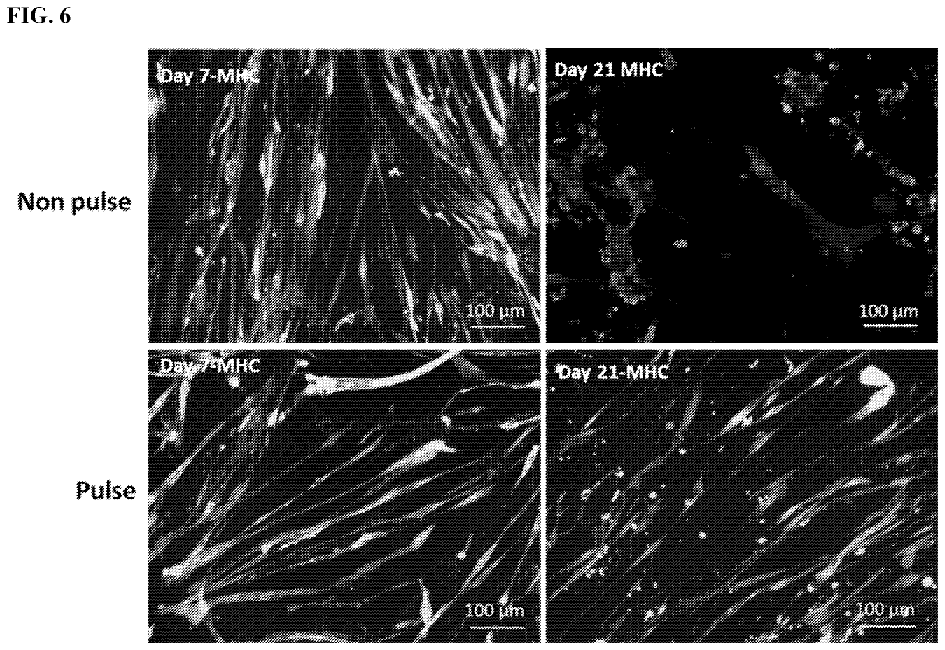

iPSC derived myoblasts obtained according to Example 1 were seeded in freshly-coated 48-well plates at density of 32×10⁶ cells/cm² in myotube differentiation medium (Stage 3 medium). Cells were either persistently cultured in Stage 3 medium (periodically replacing the medium with fresh Stage 3 medium), or “pulsed” by briefly culturing in Stage 2 medium (e.g., overnight) before returning to Stage 3 medium for another 3-4 days (with pulsing cycles repeated until the specified time point). In cells subjecting to pulsing, the first pulse was started after four days in Stage 3 medium. For cells that were not pulsed, the myotube differentiation medium was replaced with fresh medium once in every four days. FIG. 5 shows illustrative microscopy images taken at Day 6, Day 15 and Day 21 after the myotube differentiation medium was first added, and shows a significant increase in the number of myotubes in the pulse conditions by comparison to the non-pulse conditions. The myotubes were also fixed and stained for MHC at Day 7 and Day 21, as shown on FIG. 6. Cells subjected to the pulse procedure exhibited significantly increased MHC expression at Day 21.

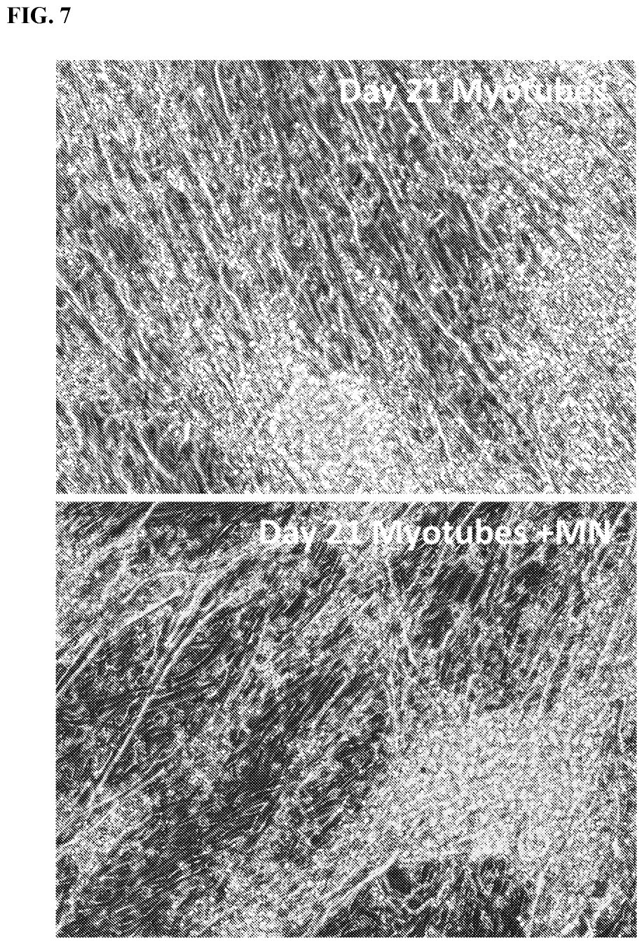

The longevity of myotubes cultured under pulsing procedure was also amenable to co-culture with motor neurons, enabling the preparation of systems for in vitro models of neuromuscular junctions. FIG. 7, shows myotubes at Day 21 that were transferred into motor neuron medium for another 7 days (top panel) and the same myotubes co-cultured with motor neurons (bottom panel). The images were taken after 7 days of culture in motor neuron medium. The motor neuron medium comprised the following components: DMEM/F12 50%, NEURALBASAL medium 50%, N2 supplement 0.5x, B27 supplement 0.5x, Ascorbic Acid 0.1 mM, GLUTAMAX 1x, All-trans Retinoic Acid 0.5 μM, Purmorphamine 0.1 μM, Compound E 0.1 μM, BDNF 10 ng/mL, CNTF 10 ng/mL, IGF-1 10 ng/mL.

References and citations to other documents, such as patents, patent applications, patent publications, journals, books, papers, and web contents, have been made throughout this disclosure. All such documents are hereby incorporated herein by reference in their entireties for all purposes.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of the various aspects of the invention disclosed herein.

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

While the present invention has been described with reference to the specific embodiments thereof, it should be understood by those skilled in the art that various changes may be made and equivalents may be substituted without departing from the spirit and scope of the invention. In addition, many modifications may be made to adapt to a particular situation, material, composition of matter, process, process step or steps, to the objective, spirit and scope of the present invention. All such modifications are intended to be within the scope of the claims appended hereto.

What is claimed is:

1. A method of differentiating pluripotent cells, the method comprising culturing pluripotent cells in a culture medium comprising one or more of a fetal bovine serum (FBS), a chick embryo extract, and a fetuin; wherein the differentiation produces myoblast precursor cells.

2. The method of claim 1, wherein the FBS is a charcoal-stripped FBS, and/or the fetuin is a bovine fetuin.

3. The method of claim 1, wherein the culture medium comprises the FBS, the chick embryo extract, and the fetuin.

4. The method of any one of claims 1-3, wherein (a) the FBS is present at a concentration of about 1% to about 10%; (b) the chick embryo extract is present at a concentration of about 0.1% to about 5%; and/or (c) the fetuin is present at a concentration of about 1 mg/mL to about 100 mg/mL.

5. The method of any one of claims 1-3, wherein the culture medium further comprises one or more of an epidermal growth factor (EGF), an insulin, dexamethasone, a ROCK inhibitor, ascorbic acid, a basic fibroblast growth factor (bFGF), a GSK3 inhibitor, or an ALK5 inhibitor.

6. The method of claim 5, wherein (a) the EGF is a human EGF, (b) the insulin is a human insulin, (c) the ROCK inhibitor is Y-27632, (d) the bFGF is a human bFGF, (e) the GSK3 inhibitor is CHIR99021, and/or (f) the ALK5 inhibitor is RepSox.

7. The method of any one of claims 1-3, wherein the pluripotent cells are induced pluripotent stem cells (iPSCs).

8. The method of any one of claims 1-3, wherein the pluripotent cells are cultured in the culture medium for about 5 to about 15 days.

9. The method of any one of claims 1-3, wherein the myoblast precursor cells comprise satellite-like cells characterized by expression of one or more of Pax3, Pax7, CD56/NCAM, Nanog, POU5F1/Oct-4, and SSEA-4.

10. The method of of any one of claims 1-3, wherein the culture medium does not comprise a horse serum.

11. The method of any one of claims 1-3, wherein the pluripotent cells are cultured on a surface pretreated with collagen I.

12. The method of any one of claims 1-3, further comprising culturing the myoblast precursor cells in a second culture medium, wherein the second culture medium comprises one or more of a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF); wherein the culturing in the second culture medium produces myoblasts.

13. The method of claim 12, wherein the myoblast precursor cells are cultured in the second culture medium for about 3 to about 14 days.

14. The method of claim 12, wherein the myoblasts are characterized by expression of one or both of MyoD and Desmin.

15. The method of claim 12, further comprising culturing the myoblasts in a third culture medium, wherein the third culture medium comprises one or more of an insulin, ascorbic acid, oncostatin, and necrosulfonamide; wherein the culturing in the third culture medium produces myotubes.

16. The method of claim 15, wherein the insulin in the third culture medium is a human insulin.

17. The method of claim 15, wherein the third culture medium comprises the insulin, ascorbic acid, oncostatin, and necrosulfonamide.

18. The method of claim 15, wherein the third culture medium comprises (a) the insulin at a concentration of about 1 μg/mL to about 25 μg/mL; (b) the ascorbic acid at a concentration of about 10 μg/mL to about 100 μg/mL; (c) the oncostatin at a concentration of about 1 ng/mL to about 100 ng/mL; and/or (d) the necrosulfonamide at a concentration of about 10 nM to about 100 nM.

19. The method of claim 15, wherein the myoblasts are cultured in the third culture medium for about 1 to about 10 days.

20. The method of claim 15, further comprising culturing the myotubes in the second culture medium.

21. The method of claim 20, wherein the myotubes are cultured in the second culture medium for about 1 to about 24 hours.

22. The method of claim 20, wherein culturing the myotubes comprises a plurality of cycles, each cycle comprising (a) culturing the myotubes in the third culture medium, and (b) culturing the myotubes in the second culture medium.

23. The method of claim 22, wherein the myotubes are maintained in culture for more than 10 days.

24. The method of claim 15, wherein the myotubes are characterized by (a) multiple nuclei, and (b) expression of one or more of myosin heavy chain, myogenin, and dystrophin.

25. Myotubes produced according to a method of claim 15.

26. A pharmaceutical composition comprising the myotubes of claim 25 and a pharmaceutically acceptable carrier.

27. Use of the myotubes of claim 25 in the production of a pharmaceutical composition.

28. Use of the myotubes of claim 25 in the production of an edible composition.

29. A culture medium for differentiating pluripotent cells, the medium comprising a fetal bovine serum (FBS), a chick embryo extract, and a fetuin.

30. The culture medium of claim 29, wherein (a) the FBS is present at a concentration of about 1% to about 10%, (b) the chick embryo extract is present at a concentration of about 0.1% to about 5%, and/or (c) the fetuin is present at a concentration of about 1 mg/mL to about 100 mg/mL.

31. The culture medium of claim 29, further comprising one or more of an epidermal growth factor (EGF), an insulin, dexamethasone, a ROCK inhibitor, ascorbic acid, a basic fibroblast growth factor (bFGF), a GSK3 inhibitor, or an ALK5 inhibitor.

32. The culture medium of claim 31, wherein (a) the EGF is a human EGF, (b) the insulin is a human insulin, (c) the ROCK inhibitor is Y-27632, (d) the bFGF is a human bFGF, (e) the GSK3 inhibitor is CHIR99021, and/or (f) the ALK5 inhibitor is RepSox.

33. The culture medium of claim 29, further comprising pluripotent cells or myoblast precursor cells.

34. The culture medium of any one of claims 29-33, wherein the culture medium does not comprise a horse serum.

35. A culture system for differentiating pluripotent cells, the culture system comprising:
(a) a first culture medium comprising one or more of a fetal bovine serum (FBS), a chick embryo extract, and a fetuin;
(b) a second culture medium comprising one or more of a horse serum, a fetuin, an EGF, an insulin, dexamethasone, a ROCK inhibitor, ascorbic acid, an ALK5 inhibitor, a hepatocyte growth factor (HGF), a bFGF, an insulin-like growth factor-1 (IGF-1), oncostatin, and a platelet-derived growth factor (PDGF); and
(c) a third culture medium comprising one or more of an insulin, ascorbic acid, oncostatin, and necrosulfonamide.

36. The culture system of claim 35, wherein the first culture medium comprises the FBS, the chick embryo extract, and the fetuin.

37. The culture system of claim 35, wherein the first culture medium comprises (a) the FBS at a concentration of about 1% to about 10%, (b) the chick embryo extract at a concentration of about 0.1% to about 5%, and/or (c) the fetuin at a concentration of about 1 mg/mL to about 100 mg/mL.

38. The culture system of claim 35, wherein the first culture medium further comprises one or more of an epidermal growth factor (EGF), an insulin, dexamethasone, a ROCK inhibitor, ascorbic acid, a basic fibroblast growth factor (bFGF), a GSK3 inhibitor, or an ALK5 inhibitor.

39. The culture system of any one of claims 35-38, wherein the second culture medium comprises the horse serum, the fetuin, the EGF, the insulin, dexamethasone, the ROCK inhibitor, ascorbic acid, the ALK5 inhibitor, the hepatocyte growth factor (HGF), the bFGF, the insulin-like growth factor-1 (IGF-1), oncostatin, and the platelet-derived growth factor (PDGF).

40. The culture system of any one of claims 35-38, wherein the third culture medium comprises the insulin, ascorbic acid, oncostatin, and necrosulfonamide.

41. The culture system of any one of claims 35-38, wherein the third culture medium comprises (a) the insulin at a concentration of about 1 μg/mL to about 25 μg/mL; (b) the ascorbic acid at a concentration of about 10 μg/mL to about 100 μg/mL; (c) the oncostatin at a concentration of about 1 ng/mL to about 100 ng/mL; and/or (d) the necrosulfonamide at a concentration of about 10 nM to about 100 nM.

42. The culture system of any one of claims 35-38, wherein the first culture medium comprises pluripotent cells or myoblast precursor cells.

43. The culture system of any one of claims 35-38, wherein the second culture medium comprises myoblast precursor cells or myoblasts.

44. The culture system of any one of claims 35-38, wherein the third culture medium comprises myoblasts or myotubes.
